(19) United States
(12) Patent Application Publication
(10) Pub. No.: US 2018/0139937 A1
(43) Pub. Date: May 24, 2018

(54) CELL CULTURE SYSTEMS FOR PRODUCING IL-33 INDUCED T9 CELLS AND METHODS OF USING THE CELLS

(71) Applicant: Indiana University Research and Technology Corporation, Indianapolis, IN (US)

(72) Inventor: Sophie Paczesny, Indianapolis, IN (US)

(21) Appl. No.: 15/571,954

(22) PCT Filed: May 6, 2016
(86) PCT No.: PCT/US2016/031310

§ 371 Date: Nov. 6, 2017

Related U.S. Application Data
(60) Provisional application No. 62/159,032, filed on May 8, 2015.
(60) Provisional application No. 62/263,185, filed on Dec. 4, 2015.

(51) Int. Cl. A01K 67/027; A61K 35/17; C12N 5/0783

(52) U.S. Cl. CPC A01K 67/0271; A61K 35/17; C12N 5/0636; C12N 2501/515; C12N 2501/2304; C12N 2501/2333; C12N 2501/51; C12N 2501/15

(57) ABSTRACT

Cell culture systems for producing IL-33 induced T9 cells and methods of using the IL-33 induced T9 cells (T9_{IL-33} cells) in a cell therapy for increasing anti-tumoral activity following allogeneic hematopoietic cell transplantation (HCT) and/or treating graft-versus-host disease (GVHD) are disclosed herein. Further, methods of using the T9_{IL-33} cells, alone or in combination with allogeneic hematopoietic cell transplantation, are described herein for cancer treatment.

### CROSS REFERENCE TO RELATED APPLICATIONS

This application claims priority to U.S. Provisional Application No. 62/263,185 filed Dec. 4, 2015 and U.S. Provisional Application No. 62/159,032 filed on May 8, 2015, both of which are hereby incorporated by reference in their entireties.

### STATEMENT OF GOVERNMENT SUPPORT

This invention was made with government support under CA168814 awarded by the National Institutes of Health. The government has certain rights in the invention.

### STATEMENT IN SUPPORT FOR FILING A SEQUENCE LISTING

A paper copy of the Sequence Listing and a computer readable form of the Sequence Listing containing the file named “IURTC_2015-100-03_ST25.txt”, which is 2,221 bytes in size (as measured in MICROSOFT WINDOWS® EXPLORER), are provided herein and are herein incorporated by reference. This Sequence Listing consists of SEQ ID NOs:1-10.

### BACKGROUND OF THE DISCLOSURE

The present disclosure relates generally to cell culture systems for producing IL-33 induced T9 cells and to methods of using the IL-33 induced T9 cells (T9_{IL-33} cells) in a cell therapy for increasing anti-tumoral activity following allogeneic hematopoietic cell transplantation (HCT). More particularly, the methods alleviate graft-versus-host disease (GVHD) severity and mortality while preserving graft-versus-leukemia (GVL) and/or graft-versus-tumor (GVT) effect. Further, these T9_{IL-33} cells are used alone or in combination with allogeneic hematopoietic cell transplantation as a cancer treatment.

Allogeneic hematopoietic cell transplantation (HCT) is a curative therapy for cancers of the bone marrow (e.g., acute myeloid leukemia (AML)). Use of HCT has increased as new techniques have allowed for transplantation in patients who previously would not have been considered HCT candidates. Approximately 30,000 allogeneic HCTs will be performed worldwide in 2020. Graft-versus-host disease (GVHD), however, remains the major contributor to morbidity and mortality for survivors of HCT. GVHD is a common complication following a bone marrow transplant from a donor. It occurs after transplant, when the donor's lymphocytes recognize parts of the patient's body as foreign. During this process, molecules (including cytokines and their receptors) are released that may damage certain body tissues, including the gut, liver and skin.

More particularly, graft-versus-tumor (GVT) reactivity relies on the recognition of alloantigens, particularly minor histocompatibility antigen (miHA) on tumor cells by donor T cells. Studies exploring the GVT effect have highlighted the ability of the human immune system to specifically and effectively eliminate cancer, and generate miHA-specific T cells that do not need gene transfer and have adequate TCR affinity. Unfortunately, T-cell reactivity to alloantigens in normal host tissues often occurs in parallel with GVT, giving rise to GVHD.

The diagnosis of GVHD currently relies on clinical symptoms and biopsies of the main target organs: skin, liver and gastrointestinal tract (GI). Some of the main effects can include red skin rash, diarrhea, sometimes with blood, and yellow jaundice. GVHD can be serious, with complications that range from mild to life threatening, even death, and often requires admission to the hospital for treatment.

Current strategies to suppress GVHD also compromise the beneficial graft-versus-leukemia (GVL) and/or graft-versus-tumor (GVT) activity.

Accordingly, there is a need in the art to develop methods for treating GVHD without compromising GVT activity. Particularly advantageous would be methods for alleviating GVHD severity and mortality while preserving graft-versus-leukemia (GVL) effect.

### BRIEF DESCRIPTION OF THE DISCLOSURE

The present disclosure is generally related to cell culture systems for producing IL-33 induced T9 cells and to methods of using the IL-33 induced T9 cells (T9_{IL-33} cells) for increasing antitumor activity and/or treating graft-versus-host disease (GVHD). More particularly, the methods alleviate GVHD severity and mortality while preserving graft-versus-leukemia (GVL) effect.

Accordingly, in one aspect, the present disclosure is directed to a cell culture system comprising interleukin 4 (IL-4), transforming growth factor beta (TGF_{β}), interleukin-33 (IL-33), antibody to cluster of differentiation 3 (anti-CD3) and antibody of cluster of differentiation 28 (anti-CD28), and a cell.

In another aspect, the present disclosure is directed to a method of cell culture for producing a T9_{IL-33} cell capable of producing cluster of differentiation 4+ (CD4+) and cluster of differentiation 8+ (CD8+) at frequencies of from about 10% to about 70% greater than a control T9 cell, the method comprising contacting a T9 cell with interleukin-33 (IL-33).

In yet another aspect, the present disclosure is directed to a method of treating graft vs. host disease (GVHD), the method comprising administering to a subject in need thereof a cellular therapy comprising T9_{IL-33} cells.

In another aspect, the present disclosure is directed to method of maintaining graft vs. leukemia activity in a subject in need thereof, the method comprising administering to the subject a cellular therapy comprising T9_{IL-33} cells.

In another aspect, the present disclosure is directed to a method of treating a cancer, the method comprising administering to a subject in need thereof a cellular therapy comprising T9_{IL-33} cells.

In yet another aspect, the present disclosure is directed to a T9_{IL-33} cell.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1E depict the effect of anti-ST2 mAb treatment on GVHD severity and mortality. FIG. 1A shows that the kinetics of plasma sST2 show increased sST2 levels in allogeneic group. Mice were treated with anti-ST2 or IgG isotype control via intraperitoneal injection on Day-1 and Day1 post-translation. FIG. 1B shows survival and clinical GVHD scores. FIG. 1C depicts that histopathologic analysis shows protection from GVHD in anti-ST2 treated group. FIG. 1D depicts increased plasma IL-22 in anti-ST2 treated group. FIG. 1E shows that anti-ST2 modulated transcript expression in MLN T cells. *p<0.05, **p<0.01, ***p<0.001, IgG vs. Anti-ST2.

FIGS. 2A & 2B depict that blockage of ST2 shifted the Th1/Th2 balance toward Th2 phenotype and increased CD4+ regulatory T cells. Spleen T cells were collected for intracellular staining at day10 post-transplantation. *p<0.05, **p<0.01.

FIGS. 3A-3F depict the ST2/IL-33 signaling effect of T9 cells. FIG. 3A depicts mST2 expression on different sorted CD4 and CD8 T cell subsets after 5 days of differentiation with indicate cytokines towards T1, T2, T9 and T9IL-33. Gene expression by real time PCR and protein level measured by flow cytometry (n=5, mean±SEM). FIG. 3B depicts a representative histogram of PU.1 expression on different subsets of T cells. FIG. 3C shows representative plots of IFNγ and IL-9 intercellular expression of in vitro differentiated cells (n=5, mean±SEM). FIG. 3D depicts the secretion of cytokine signature of different T cell subsets (IFNγ, IL-4, IL-9 and IL-10) measured by ELISA (n=4, mean±SEM). FIG. 3E shows clinical scores of GVHD and survival curves for lethally irradiated BALB/c mice. The mice (900 cGy) were transplanted with B6 or syngeneic BM cells (5×10⁶) and 1×10⁶ in vitro differentiated splenic T into T0, T1, T2 T9 and T9_{IL-33}. FIG. 3F shows clinical scores of GVHD and survival curves in comparison to adoptive transfer of T9_{IL-33} generated from ST2^{-/-} or IL9^{-/-}.

FIGS. 4A & 4B depict the effects of IL-33/ST2 signaling on cytolytic molecule expression. FIG. 4A shows that CD4 and CD8 T cells purified from IL-33 induced T9 from WT mice had higher expression of Granzyme A than IL33-induced T9 from ST2−/− and IL-9−/− mice. FIG. 4B is a cytolytic assay showing tumor cell viability in CD4 and CD8 T cells purified from IL-33 induced T9 from WT mice.

FIG. 5 shows that ST2 and IL-9 signaling are required to improve T9 GVT activity.

FIGS. 6A & 6B depict the effects of administering T9_{IL-33} cells in a cell therapy on tissue damage resulting from GVHD (co-culture of T9_{IL-33} cells with colonic epithelial cells (FIG. 6A) and through a transwell co-culture showing that the effect is contact dependent (FIG. 6B)).

FIGS. 6C & 6D are an ex vivo analysis of IFN-γ expression in CD4 and CD8 T cells from GVHD target organs in mice that received T9_{IL-33}, or T9_{IL-33} from ST2^{-/-} or T9_{IL-33} from IL9^{-/-}, showing the decreased IFN-γ expression by T cells in target organs when T9_{IL-33} were transferred. This effect was abolished when T9_{IL-33} from ST2^{-/-} or from IL9^{-/-} are transferred.

FIGS. 7A-7E depict the transcriptome and phenotype of T9_{IL-33} cells generated from WT versus ST2^{-/-}. Molecules implicated in anti-leukemic activity are upregulated (e.g., GrazA, GrazB, CD160, KLRK1) as well as activation markers of central memory (e.g., CD69, CD27).

FIGS. 8A & 8B depict cytolytic assays against MLL-AF9 leukemic cells of allogeneic T9_{IL-33} cells generated from WT T cells or from ST2−/− T cells, with CD4 sorted T cells (FIG. 8A) or CD8 sorted T cells (FIG. 8B).

FIG. 8C depicts survival curves for lethally irradiated BALB/c mice. The mice that were injected with MML-AF9 leukemia were transplanted with B6 or syngeneic BM cells (5×10⁶) and 1×10⁶ in vitro differentiated splenic T into T1, T9 and T9IL-33.

FIG. 8D depicts the same GVL model as FIG. 8C with adoptive transfer of T9_{IL-33} cells generated from ST2−/− or IL9−/−.

FIG. 8E shows a transcriptome analysis of T9_{IL-33} from WT versus ST2−/− (same transcriptome analysis of FIG. 7D), which showed CD8α transcript was upregulated in both CD4 and CD8 T cells.

FIG. 8F depicts confirmation of the transcriptome analysis of FIG. 8E at the protein level with expression of CD8α.

FIG. 8G depicts the effects of blocking CD8α with a neutralizing antibody during T9_{IL-33} differentiation. Particularly, blocking CD8α reduced the cytotoxicity of both murine T9_{IL-33} and human T9_{IL-33} cells as compared to the isotype control.

FIGS. 9A-9G depict the effects of ST2/IL-33 signaling on T9 cells in vitro and in vivo. FIG. 9A depicts mST2 expression on sorted CD4 and CD8 T-cell subsets after 5 days of differentiation. mRNA expression was measured by real-time PCR and protein expression by flow cytometry (isotype control, T1, T2, T9, and T9_{IL-33}; n=5, mean±SEM). FIG. 9B shows representative plots of IFN-γ and IL-9 expression from in vitro differentiated cells and a bar graph showing the frequency of IL-9-expressing T cells (n=4, mean±SEM). FIG. 9C depicts secretion of signature cytokines from different T-cell subsets (n=4, mean±SEM). FIG. 9D shows a representative histogram of PU.1 expression on T-cell subsets from 5 independent experiments (isotype control, T1, T2, T9, and T9IL-33). FIG. 9E depicts clinical scores of GVHD and survival curves for BALB/c mice transplanted with B6 or syngeneic bone marrow (BM) cells and in vitro differentiated or syngeneic T cells (syngeneic, T0, T1, T2 T9 and T9_{IL-33}; n=12 each group). FIG. 9F depicts clinical scores of GVHD and survival curves for BALB/c receiving B6 or syngeneic BM cells and in vitro differentiated or syngeneic T cells (syngeneic, WT T9_{IL-33}, ST2^{-/-}T9_{IL-33}, or IL-9^{-/-}T9_{IL-33} cells; n=24 per group). FIG. 9G depicts clinical scores of GVHD and survival curves for C3H.SW mice receiving B6 or syngeneic BM cells and in vitro differentiated or syngeneic T cells (syngeneic, WT T9_{IL-33}, ST2^{-/-}T9_{IL-33}; n=7). For FIGS. 9E-9G, p values were calculated for GVHD scores by t test and for survival by Log-rank test. **p<0.01; ***p<0.001.

FIGS. 10A-10C depict the impact of CD4 T cells on CD8 T cells during T9_{IL-33} differentiation. Splenic CD4 and CD8 cells were purified by microbeads and either co-cultured together or separated in a Transwell with anti-CD3/CD28, IL-4, TGF-β and IL-33 for 5 days. FIGS. 10A & 10B show IL-9 and PU.1 expression on CD8 T cells from co-cultures with or without Transwells. FIG. 10C depicts IL-9 secretion from total T9_{IL-33} co-culture (Co) or through Transwell (TW). Data represent 3 independent experiments. *p<0.05; **p<0.01; ***p<0.001, as calculated by t-test.

FIG. 11 depicts the impact of T1 vs T9_{IL-33} cells on gut pathology. Pathology index of mouse intestines at day 10 after allo-HCT with either T1 or T9_{IL-33} cells (n=3). *p<0.05, as calculated by Mann-Whitney U test.

FIGS. 12A-12J depict mechanisms of T9_{IL-33} cell protection of gut epithelial cells. FIG. 12A are representative plots of Ki67 staining in gut T cells collected from mice on day 10 after all-HCT with syngeneic BALB/c T cells or allogeneic in vitro differentiated T cells. FIG. 12B depict absolute counts of gut-infiltrating T cells in the same mice as in FIG. 12A. FIG. 12C depicts transcriptome analysis of I120 and Cd160 in sorted WT T9_{IL-33} vs ST2^{-/-}T9_{IL-33} CD4 and CD8 T cells. FIG. 12D depicts AREG expression in in vitro differentiated and sorted CD4 subsets. Gene expression was measured by real-time PCR and protein level by flow cytometry (n=3). FIG. 12E depicts EGFR gene expression in intestinal stem cells and epithelial cells from gut of naïve BALB/c mice. FIG. 12F depicts the percentage of dead BALB-5047 cells after co-culture with T1, WT T9_{IL-33} or ST2^{-/-}T9_{IL-33} cells for 6 hours in the presence of anti-AREG blocking antibody or isotype control (n=3). FIG. 12G depicts AREG expression in sorted CD4 subsets from intestine of GVHD mice collected on day 14 after allo-HCT with T1, WT T9_{IL-33} or ST2^{-/-}T9_{IL-33} cells. Gene expression was measured by real-time PCR and protein level by flow cytometry (n=4). FIG. 12H depicts ex-vivo expression of IFN-γ and IL-17 in gut CD4 T cells collected on day 14 after allo-HCT with allogeneic T1, WT T9_{IL-33} or ST2^{-/-}T9_{IL-33} cells (n=4). FIG. 12I depicts AREG expression on in vitro differentiated human T1, T9 and T9_{IL-33}CD4 cells from healthy donors (n=3). FIG. 12J depicts the percentage of dead human normal colonic cells after co-culture with T1, T9 and T9_{IL-33} cells for 6 hours in the presence of anti-AREG blocking antibody or isotype control (n=3). *p<0.05; **p<0.01; ***p<0.001, as calculated by t test.

FIGS. 13A-13E depicts the effect of ST2/IL-33 signaling on gut T-cell proliferation, viability and migration, Treg expansion and ILC2s. Lethally irradiated BALB/c mice received 10⁶ B6 CFSE-labelled T1, WT T9_{IL-33} or ST2^{-/-}T9_{IL-33} cells together with 5×10⁶ WT BM cells. FIG. 13A are representative plots of CFSE dilution for gut-infiltrating T cells on day 5 post-HTC (n=3). FIG. 13B are representative plots of annexin V and viability dye staining of gut T cells at day 10 post-HTC (n=3). FIG. 13C are representative plots of cx4β7 and CRK (top) and CCR5 (bottom) in CD4 T cells infiltrating the gut at day 10 post-HTC (n=3). FIG. 13D are representative plots of CD4 and FoxP3 and a bar graph showing the frequency of Tregs (CD4⁺FoxP3⁺) in gut-infiltrating CD4 T cells at day 10 post-HTC (n=4). FIG. 13E are representative plots of mST2 and Gata3 in Lin-CD45⁺CD90.2⁺ cells (ILC2 markers, n=4). p<0.05; **p<0.01; ***p<0.001, as calculated by t-test.

FIGS. 14A-14F depict allogeneic T cell interaction with colonic epithelial cells. FIG. 14A depicts B6 T1, WT T9_{IL-33} or ST2^{-/-}T9_{IL-33} cells differentiated in MLR conditions that were co-cultured with BALB-5047 colonic epithelial cells together (left) or through Transwells (right) for 6 hours. Percentage of dead BALB-5047 cells was measured by viability dye staining and flow cytometry. FIG. 14B depicts the percentage of dead BALB-5047 cells co-cultured with T1, WT T9_{IL-33} or ST2^{-/-}T9_{IL-33} cells in the presence of anti-IL-20Rb or isotype control for 6 hours. FIG. 14C are representative plots of CD160 expression on WT and ST2^{-/-}T9_{IL-33} CD8 cells. FIG. 14D is a histogram showing HVEM (CD160 ligand) expression on BALB-5047 cells. FIG. 14E depicts the percentage of dead BALB-5047 cells co-cultured with T1, WT T9_{IL-33} or ST2^{-/-}T9_{IL-33} cells in the presence of anti-HVEM or isotype control for 6 h. *p<0.05; **p<0.01; ***p<0.001, as calculated by t-test. FIG. 14F are representative plots of ISC and epithelial cell staining with Lgr5 (monoclonal antibody from R&D Systems, clone #889901) and EpCam, gated on live, single, CD45− cells from gut of naïve BALB/c mice.

FIG. 15A depicts the effect of ST2/IL-33 signaling on human T9 cells. mST2 expression on human CD4 and CD8 T-cell subsets after 7 days of differentiation (isotype control, T1, T2, T9, and T9_{IL-33}, n=4).

FIG. 15B are representative plots of IL-9 and IFN-γ expression on human T cells differentiated into T9 cells in the presence or absence of IL-33, and a bar graph showing the frequency of IL-9expressing T cells (n=4). IL-9 secretion from total T9 or T9IL-33 (n=3) *p<0.05, **p<0.01, as calculated by t-test.

FIGS. 16A-16G depict T9_{IL-33} cells and anti-tumor activity. FIG. 16A are survival curves for BALB/c mice receiving 104 syngeneic MLL-AF9 leukemic cells with syngeneic T cells or allogeneic in vitro differentiated cells (syngeneic, T1, T9, WT T9_{IL-33}, ST2^{-/-}T9_{IL-33}, IL-9^{-/-}T9_{IL-33}; n=12 mice per group). ***p<0.0001 by Log-rank test. Pie charts show cause of death (Tumor, GVHD, No death). FIG. 16B depicts transcriptome analysis of Gzma, Gzmb, Prf1, Cd621, Tcf7, Cd27, and Fas expression in sorted WT T9_{IL-33} versus ST2^{-/-}T9_{IL-33} CD4 and CD8 cells. FIG. 16C are representative plots of granzyme B and perforin expression in WT T9_{IL-33} and ST2^{-/-}T9_{IL-33} cells gated on CD8, and bar graphs showing the frequency of granzyme B⁺ and perforin⁺ T cells (n=4). FIG. 16D depicts cytolytic assays: B6 WI I9_{IL-33}, B6 ST2^{-/-}_{T9IL-33} or C3H.SW T9_{IL-33} mixed lymphocyte reaction (MLR) cultures were co-cultured with C3H.SW-derived MLL-AF9 cells for 6 hours (WT T9_{IL-33}, ST2^{-/-}T9_{IL-33}, syngeneic T9_{IL-33}; n=4). FIG. 16E are representative plots of granzyme B and perforin expression in gated CD8 I cells from BM 28 days after adoptive transfer of allogeneic I cells with syngeneic MLL-AF9 cells. Bar graphs show the frequency of granzyme B⁺ and perforin⁺ CD8 T cells. FIG. 16F are representative plots of CD62L⁺ and CD44⁺ (top) and CD27⁺ and KLRG1⁺ (bottom) cells, and bar graphs showing the frequency of CD44⁺CD62L⁺ and CD27⁺ CD8 T cells from in vitro differentiated cells from WT T9_{IL-33} versus ST2^{-/-}T9_{IL-33} cells (n=4). FIG. 16G are representative plots of CD62L⁺ and CD44⁺ (top) and CD27⁺ and KLRG1⁺ (bottom) cells, and bar graphs showing the frequency of CD44⁺CD62L⁺ and CD27⁺ CD8 T cells from BM collected on day 28 from mice receiving MLL-AF9 leukemic cells with WT T9_{IL-33} or ST2^{-/-}T9_{IL-33} cells (n=4) *p<0.05; **p<0.01; ***p<0.001, as calculated by t test.

FIGS. 17A & 17B depict T9_{IL-33} cells and anti-tumor activity. FIG. 17A are survival curves for BALB/c mice receiving 0.2×10⁶ cells of the syngeneic A20 lymphoma cell line with syngeneic T cells or allogeneic in vitro differentiated cells (syngeneic, T1, T9, WT T9_{IL-33}, ST2^{-/-}T9_{IL-33}, IL-9^{-/-}T9_{IL-33}; n=12 mice per group). ***p<0.0001, by Log-rank test. FIG. 17B are survival curves for C3H.SW mice receiving 104 MLL-AF9 leukemic cells with syngeneic T9_{IL-33} cells or allogeneic in vitro differentiated cells (syngeneic T9_{IL-33}, WT T9_{IL-33}, ST2^{-/-}T9_{IL-33}; n=14 mice per group). ***p<0.0001, by Log-rank test.

FIGS. 18A-18F depict that ST2/IL-33 signaling on CD4 impacts CD8 anti-tumor activity. FIG. 18A depicts Granzyme B and perforin expression on CD8 T cells cultured with WT or ST2^{-/-} CD4 cells together or through a Transwell under T9_{IL-33} conditions. FIG. 18B are cytolytic assays of sorted CD4 and CD8 cells from C3H.SW T9_{IL-33}, B6 WT T9_{IL-33} or B6 ST2^{-/-}T9_{IL-33} cells incubated for 6 hours with C3H.SW MLL-AF9 cells (C3H.SW T9_{IL-33}, WT T9_{IL-33}, ST2^{-/-}T9_{IL-33}; n=4). FIG. 18C are cytolytic assays of purified CD8 cells differentiated into T9_{IL-33} cells alone or in the presence of CD4 in MLR conditions (in presence of CD4, CD8 alone; n=4). FIG. 18D depict mRNA expression of Egfr on BALB-5047, MLL-AF9 cells by qPCR. FIG. 18E depict synergenic T9_{IL-33}, WT T9_{IL-33} or ST2^{-/-}T9_{IL-33} cells that were differentiated in MLR conditions and co-cultured with BALB/c MLL-AF9 cells for 6 hours at a ratio of 10:1 with anti-AREG. FIG. 18F depict KLRG1 expression on CD8 cells cultured together or through a Transwell with CD4 T cells. p<0.05; **p<0.01; ***p<0.001, as calculated by t-test.

FIGS. 19A-19H depict the mechanisms of T9_{IL-33} cell killing of tumor cells. FIG. 19A depict transcriptome analysis of CD8α expression on sorted WT T9_{IL-33} vs ST2^{-/-}T9_{IL-33} CD4 and CD8 cells. FIG. 19B are representative plots of CD8α expression on CD4⁺ and CD8β⁺ T cells from in vitro differentiated WT T9_{IL-33} and ST2^{-/-}T9_{IL-33} cells (n=4). FIG. 19C are cytolytic assays: B6 T9_{IL-33} cells were differentiated in MLR conditions with anti-CD8α blocking antibody or isotype control. After 5 days, T9_{IL-33} cells were incubated with BALB/c MLL-AF9 cells for 6 hours (isotype, anti-CD8α, n=3). FIG. 19D are cytolytic assays of in vitro differentiated B6 T9_{IL-33} incubated with BALB/c MLL-AF9 cells in the presence of anti-CD8α or isotype control for 6 hours (isotype, anti-CD8α, n=3). FIG. 19E depicts ImageStream cell images of syngeneic T9_{IL-33} or allogeneic T9_{IL-33} cells incubated with BALB/c eGFP-MLL-AF9 cells and anti-CD8α blocking antibody or isotype control for 3 hours. FIG. 19F are representative plots of human granzyme B and granzyme K expression on T9 and T9_{IL-33} cells, and bar graphs showing the frequencies of granzyme B⁺ and granzyme K⁺ cells (n=3). FIG. 19G are cytolytic assays of human T9 or T9_{IL-33} cells incubated for 6 hours with MOLM14 leukemia cells (T9, T9_{IL-33}, n=3). FIG. 19H are cytolytic assays of human T9_{IL-33} cells differentiated with anti-CD8α blocking antibody or isotype control and incubated with MOLM14 cells for 6 hours (isotype, anti-CD8α, n=3). *p<0.05; **p<0.01; ***p<0.001, as calculated by t-test.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Definitions

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be blood or any blood constituents (e.g., whole blood, plasma, serum) from the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

The biological sample used in the methods of the present disclosure can be obtained using certain methods known to those skilled in the art. Biological samples may be obtained from vertebrate animals, and in particular, mammals. In certain instances, a biological sample is whole blood, plasma, or serum.

As used herein, the terms “control”, “control cohort”, “reference sample”, “reference cell”, “reference tissue”, “control sample”, “control cell”, and “control tissue” refer to a sample, cell or tissue obtained from a source that is known, or believed, to not be afflicted with the disease or condition for which a method or composition of the present disclosure is being used to identify. The control can include one control or multiple controls. In one embodiment, a reference sample, reference cell, reference tissue, control sample, control cell, or control tissue is obtained from a healthy part of the body of the same subject or patient in whom a disease or condition is being identified using a composition or method of the present disclosure. In one embodiment, a reference sample, reference cell, reference tissue, control sample, control cell, or control tissue is obtained from a healthy part of the body of an individual who is not the subject or patient in whom a disease or condition is being identified using a composition or method of the invention.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition (such as GVHD, cancer and/or a hematological malignancy) or be diagnosed with a condition (such as GVHD, cancer and/or a hematological malignancy). The subject can also be suspected of having or being at risk for having GVHD, cancer and/or a hematological malignancy. According to one embodiment, the subject to be treated according to this present disclosure is a human.

As used herein, “treating”, “treatment”, “alleviating”, “alleviate”, and “alleviation” refer to measures, wherein the object is to prevent or slow down (lessen) the targeted pathologic condition or disorder or relieve some of the symptoms of the disorder. Those in need of treatment can include those already with the disorder as well as those prone to have the disorder, those at risk for having the disorder and those in whom the disorder is to be prevented.

As used herein, “maintain”, or “maintaining” refer to measures, wherein the object is to preserve or sustain a particular function or activity.

“Elevated expression level” and “elevated levels” refer to an increased expression of an mRNA or a protein in a patient (e.g., a patient suspected of having or diagnosed as having GVHD, cancer and/or a hematological malignancy) relative to a control, such as a subject or subjects who are not suffering from GVHD, cancer and/or a hematological malignancy. Levels can be determined using any methods known in the art, for example, Western blot, Southern blot, PCR, Northern blot, immunoprecipitation, ELISA, mass spectrometry, and like.

The present disclosure is generally directed to cell culture systems for producing IL-33 induced T9 cells and to methods of using the IL-33 induced T9 cells (T9_{IL-33} cells) for treating graft-versus-host disease (GVHD). More particularly, the methods alleviate GVHD severity and mortality while preserving graft-versus-leukemia (GVL) and/or graft-versus-tumor (GVT) effect. Generally, the cell culture system includes the combination of interleukin 4 (IL-4), transforming growth factor beta (TGFβ), interleukin-33 (IL-33), antibody to cluster of differentiation 3 (anti-CD3) and antibody to cluster of differentiation 28 (anti-CD28) with a cell.

In one particular embodiment, the cell culture system includes from about 5 ng/ml to about 100 ng/ml, and including about 20 ng/ml, IL-4, from about 1 ng/ml to about 10 ng/ml, and including about 4 ng/ml, TGFβ, from about 5 ng/ml to about 100 ng/ml, and including about 10 ng/ml, IL-33, from about 1 μg/ml to about 10 μg/ml anti-CD28, and from about 0.5 μg/ml to about 5 μg/ml anti-CD3.

The cell may be a peripheral blood mononuclear cell (PBMC) or a spleen cell. In one embodiment, the cell is a peripheral blood mononuclear cell (PBMC), such as a lymphocyte, and in particular, a T cell. Suitable T cells include a cluster of differentiation 4+ (CD4+) T cell and a cluster of differentiation 8+ (CD8+) T cell. In one embodiment, the T cell is a T helper (Th9) cell. In another embodiment, the T cell is a T cytotoxic 9 (Tc9) cell.

In one aspect of the present disclosure, the cell culture system is used to produce a IL-33 induced T9 cell (T9_{IL-33} cell) capable of producing cluster of differentiation 4+ (CD4+) and cluster of differentiation 8+ (CD8+) at frequencies of from about 10% to about 70% greater than a control T9-cell (i.e., a T9-cell not cultured in the cell culture system of the present disclosure, and thus, not IL-33 induced). The methods of using the cell culture system allow for contacting a PMBC cell, such as a T9-cell, with interleukin-33 (IL-33). In one embodiment, the T9-cell is contacted with from about 5 ng/ml to about 100 ng/ml IL-33, including about 10 ng/ml, IL-33.

In one embodiment, the methods of using the cell culture system produce IL-33 induced T9 cells, including IL-33 induced T helper (Th9) cells and IL-33 induced T cytotoxic 9 (Tc9) cells.

It has been found that the T9_{IL-33} cells have increased expression of Suppression of Tumorigenicity2 (ST2) and Spi-1 Proto-Oncogene (referred to herein as PU.1) as compared to control T9-cells.

Further, the T9_{IL-33} cells express cell surface markers such as cluster of differentiation 8α (CD8α), suppression of tumorigenicity2 (ST2), interleukin-9 (IL-9), interleukin-10 (IL-10), Granzyme A (GrazA), Granzyme B (GrazB), cluster of differentiation 160 (CD160), Killer Cell Lectin-Like Receptor Subfamily K, Member 1 (KLRK1), cluster of differentiation 69 (CD69), cluster of differentiation (CD27), L-selectin (CD62L), CD45RO, CD45RA, Chemokine (C-C Motif) Receptor 7 (CCR7), and combinations thereof.

It was found that the T9_{IL-33} cells do not express interferon-gamma (IFNγ) or interleukin-4 (IL-4).

In another aspect of the present disclosure, the T9_{IL-33} cells prepared in the present disclosure can be used in cell therapy for treating disorders and conditions. For example, in one embodiment, the T9_{IL-33} cells can be administered to a subject in need thereof for treating graft vs. host disease (GVHD). In another embodiment, the T9_{IL-33} cells can be administered to a subject in need thereof for treating a solid tumor cancer, such as melanoma, breast cancer, prostate cancer, lung cancer, pancreatic cancer, and the like. In yet another embodiment, the T9_{IL-33} cells can be administered to a subject in need thereof for treating a hematological malignancy, such as leukemia (e.g., acute myeloid leukemia (AML), acute lymphoblastic leukemia (ALL) from B and T origin, myeloid/lymphoid or mixed-lineage leukemia (MLL)), and myeloma.

In yet another aspect of the present disclosure, the T9_{IL-33} cells prepared in the present disclosure can be used for maintaining graft vs. leukemia activity (GVL) in a subject in need thereof.

In some embodiments, the T9_{IL-33} cells are administered as part of a pharmaceutical composition including the T9_{IL-33} cells admixed with a physiologically compatible carrier. As used herein, “physiologically compatible carrier” refers to a physiologically acceptable diluent such as water, phosphate buffered saline, or saline, and further may include an adjuvant.

The pharmaceutical compositions including the combination of cells and physiologically compatible carriers used in the methods of the present disclosure can be administered to a subset of subjects in need of treatment for GVHD, cancer, and/or hematological malignancy. Some subjects that are in specific need of treatment for GVHD, cancer, and/or hematological malignancy may include subjects who have or are susceptible to, or at elevated risk of, experiencing GVHD, cancer, and/or hematological malignancy, and the like. Subjects may be susceptible to, or at elevated risk of, experiencing GVHD, cancer, and/or hematological malignancy due to family history, age, environment, and/or lifestyle. For example, subject has received allogeneic hematopoietic cell transplantation (allo-HCT) are at risk of GVHD. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

The disclosure will be more fully understood upon consideration of the following non-limiting Examples.

### EXAMPLES

### Example 1

In this Example, the effects of blocking soluble suppressor of tumorigenicity 2 (ST2) were analyzed.

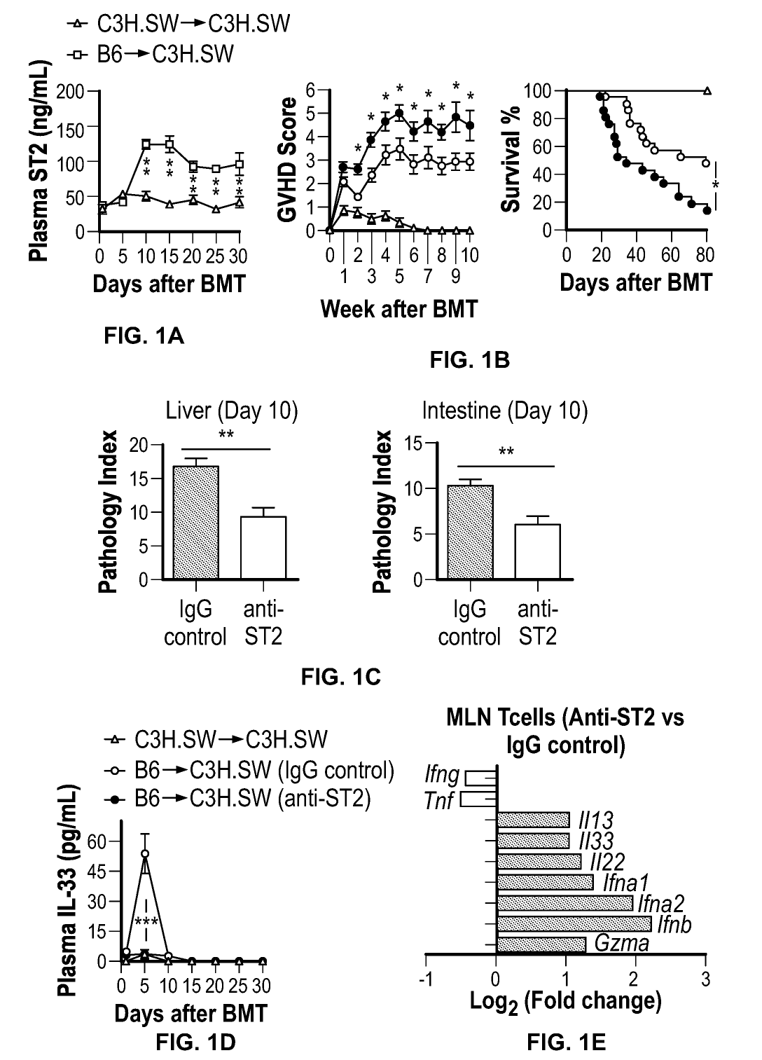

ST2 is a member of the IL-1 receptor family whose sole known ligand is IL-33. Soluble ST2 acts as a decoy receptor for IL-33. First, plasma levels of ST2 HCT patients were determined. As shown in FIG. 1A, It was found that, plasma ST2 was markedly increased prior to and at the onset of GVHD symptoms in multiple clinically relevant GVHD murine models (B6 to C3H.SW shown). Based on this observation, anti-ST2 antibody was given with the following prophylactic schedule: one dose before HCT and one dose at day+1 post-HCT). As shown in FIG. 1B, dosing with anti-ST2 antibody significantly reduced GVHD severity and mortality. Further, pathology analysis indicated that anti-ST2 treated recipients showed lower histopathologic score in liver and intestine (FIG. 1C). Strikingly, anti-ST2 significantly increased plasma IL-33 (FIG. 1D).

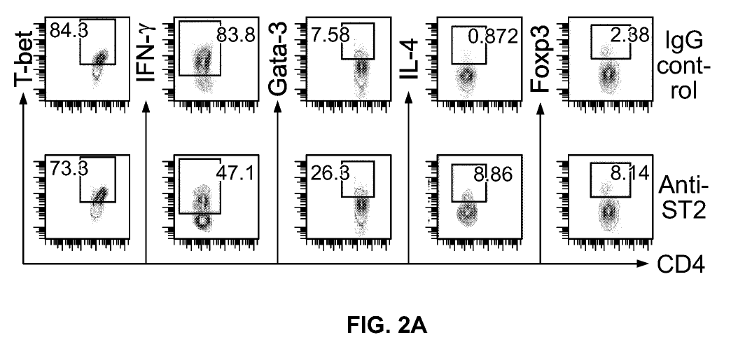

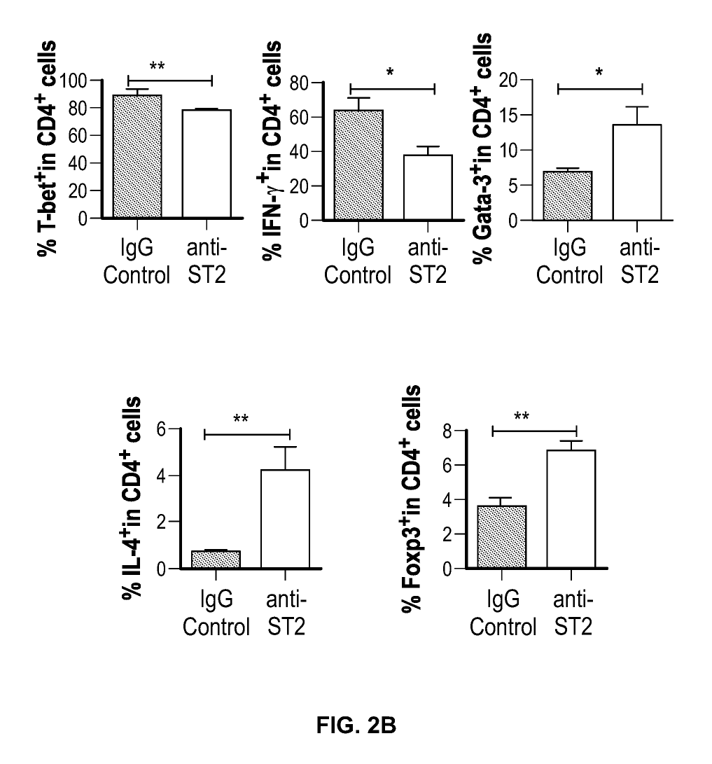

Additionally, whole transcriptome analysis of mesenteric lymph node T cells showed that anti-ST2 modulated gene expression of helper T cell (Th1/Th2) related cytokines (FIG. 1E), suggesting that ST2 blockade may affect the helper T cell compartment. To further assess the effects of ST2 blockade on helper T cells compartment, the Th1/Th2 balance was examined by flow cytometry. As shown in FIGS. 2A & 2B, administration of anti-ST2 shifted Th1/Th2 balance toward a Th2 phenotype and also induced expansion of T_{reg}.

### Example 2

In this Example, mouse T9_{IL-33} cells were prepared using methods of the present disclosure.

Particularly, T cells from splenocytes of C57BL/6 wild type mice were purified using mouse Pan T cell kit (Miltenyi Biotec, Germany). The cells were resuspended in a concentration of 1×10⁶ T cells/ml in complete DMEM media (10% FBS, 1% PS, 1% L-glu, 1% HEPES, 1% non-essential amino acids, 0.1% 2ME). Cytokines were added for differentiation (20 ng/ml mIL-4, 4 ng/ml mTGF-β, +/−10 ng/ml mIL-33)+5-10 μg/ml anti-CD28. Cells were plated in pre-coated flat bottom plates for 2-4 hours with 1 μg/ml anti-CD3 (50 μl for 96-well plate, 100 μl for 48-well plate, 200 μl for 24-wells plate at 37° C.).

### Example 3

In this Example, the effects of ST2/interleukin 33 (IL-33) activation of interleukin 9 (IL-9) secreting T cells on fatal immunity and tumor immunity were analyzed.

Elevated IL-33 sequestering soluble ST2 in the plasma has been shown to be a risk factor for severe GVHD. In addition, the IL-9-secreting Th9 and Tc9 cell subsets have higher antitumor activity than Th1 and Tc1. In this Example, T9 cells were differentiated in vitro in the presence or absence of IL-33.

Total T cells from C57BL/6 wild type, ST2−/− mice, or IL-9−/− mice were cultured as described in Example 1 with anti-mouse CD3 and anti-mouse CD28 in the presence of recombinant IL-4 and TGF-β or additional IL-33 for 5 days for T9 conditions.

In parallel, T cells were polarized towards T1 and T2 in the presence of IL-12 and IL-4, respectively. Cells were collected for flow cytometry analysis.

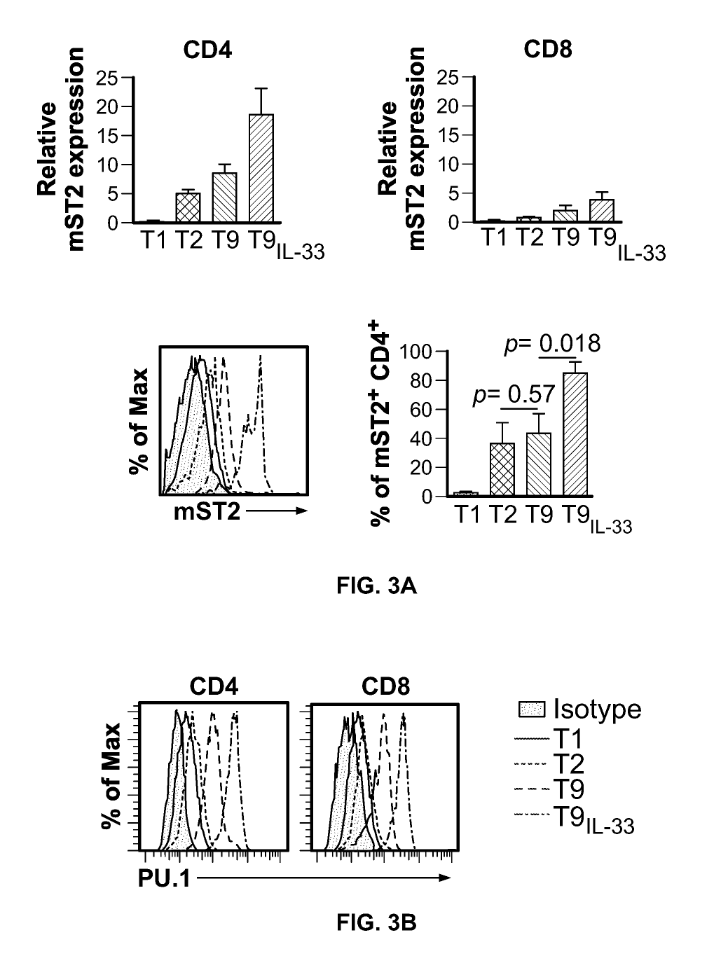

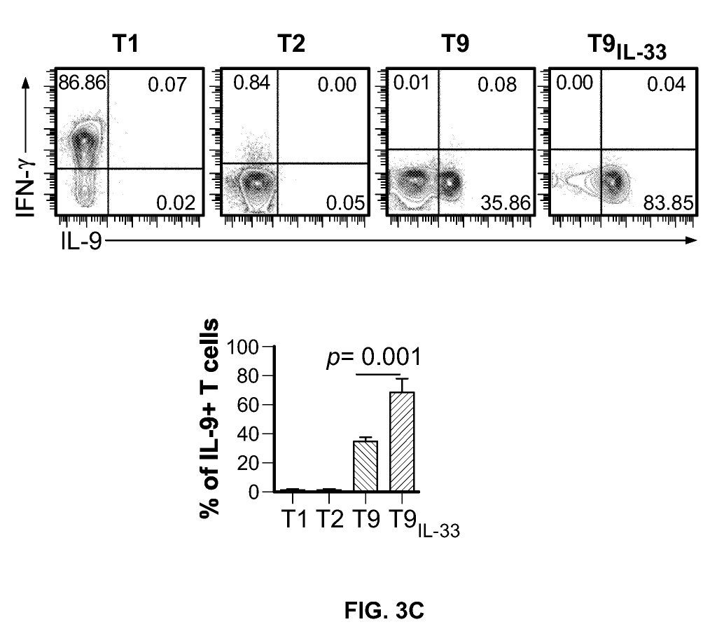

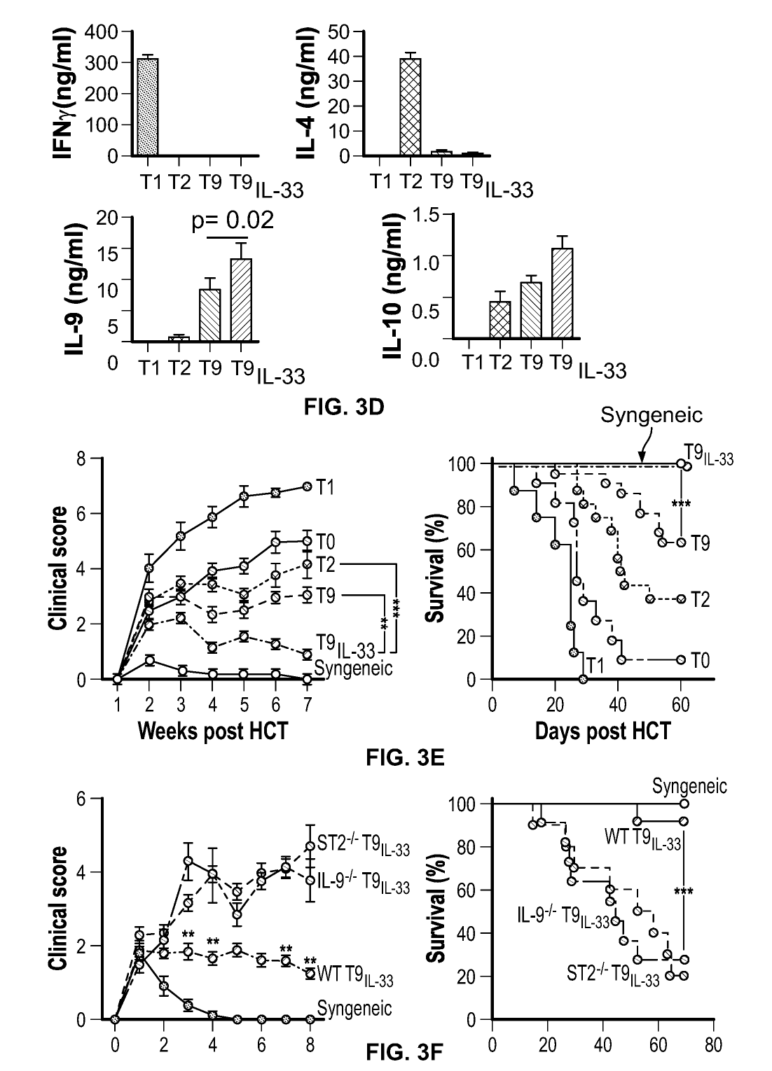

Polarized T cells were subjected to surface and intracellular staining for ST2 and PU.1, respectively. As shown in FIGS. 3A and 3B, addition of IL-33 enhanced the expression of mST2 on CD4 and PU.1 on both CD4 and CD8 T cells. Particularly, as shown, 50% of T9 cells expressed mST2 and differentiation of total T cells into T9 cells in the presence of IL-33 (i.e., T9_{IL-33} cells) increased expression of mST2 (FIG. 3A) and PU.1 (FIG. 3B), a transcription factor that promotes IL-9 production on both CD4 and CD8 T cells.

Further, as shown in FIGS. 3C and 3D, the addition of IL-33 significantly enhanced IL-9 expression and secretion on T9 cells, without any expression of IFNγ or IL-4, the signature of T1 and T2 cells, respectively. Additionally, IL-10 production level was increased (FIG. 3D).

Further, as shown in FIG. 3E, mice receiving T9 cells developed very mild and significantly less disease than those receiving T2 cells up to 60 days post HCT. And, as shown in FIG. 3F, mice receiving IL-33 induced T9 cells generated from ST2−/− or IL-9−/− donors developed significantly more severe disease and had higher mortality than those receiving T9 cells from wild-type (WT) donors. These results confirm that the addition of IL-33 to the T9 condition further increased protection against GVHD.

### Example 4

In this Example, the effects on induction of the ST2/IL-33 pathway on GVT activity were analyzed.

Recipient Balb/C mice received bone marrow cells and T cells as described in Example 2 above with or without 1×10⁴ of GFP+MLL-AF9 AML cells. Mice were followed daily for survival and mortality due to GVHD or tumor.

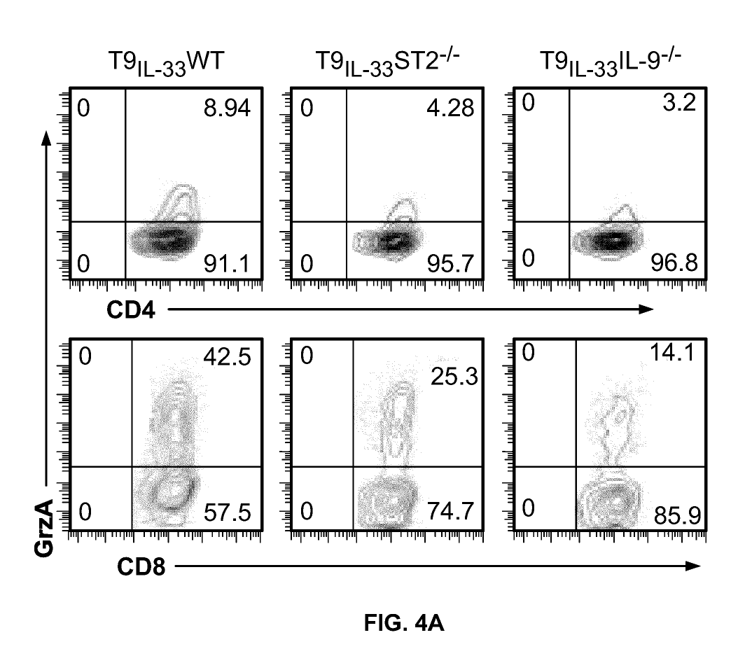

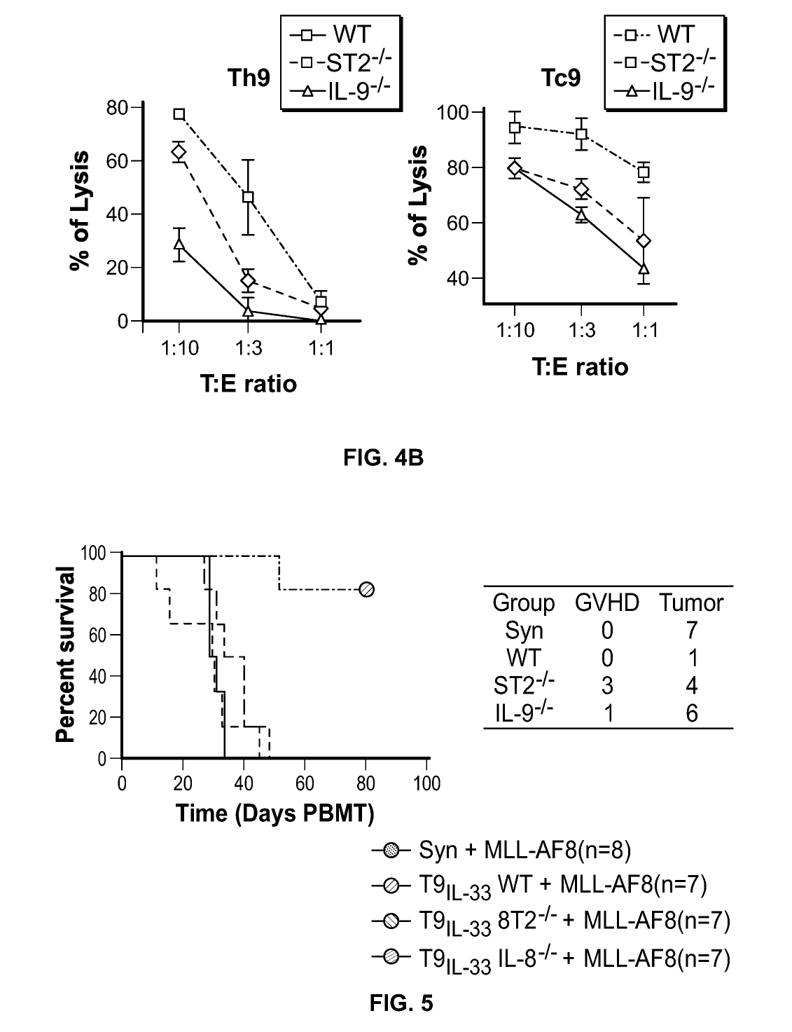

As shown in FIG. 4A, CD4 and CD8 T cells purified from IL-33 induced T9 from WT mice had higher expression of Granzyme A than IL33-induced T9 from ST2−/− and IL-9−/− mice. A cytolytic assay in vitro confirmed these observations, using the A20 lymphoma cell line co-cultured with IL-33 induced T9 from WT or ST2−/− and IL-9−/− mice (FIG. 4B). Tumor cell viability was determined by flow cytometry.

Additionally, as shown in FIG. 5, mice receiving IL-33 induced T9 cells generated from ST2−/− or IL-9−/− donors had less GVT activity compared to WT donor T cells and died of tumor within 50 days post-HCT.

### Example 5

In this Example, human T9_{IL-33} cells were prepared using methods of the present disclosure.

Particularly, T cells from human PBMCs were purified using human Pan T cell kit (Miltenyi Biotec, Germany). The cells were resuspended in a concentration of 1×10⁶ T cells/ml in complete RPMI media (10% HS, 1% PS, 1% L-glu, 1% HEPES, 1% non-essential amino acids, 0.1% 2ME). Cytokines were added for differentiation (20 ng/ml hIL-4, 4 ng/ml hTGF-β, +/−10 ng/ml hIL-33). Cells were plated in round bottom plates (2-4 hours), 200 μl for 96 wells plate at 37° C. Anti-CD3 and anti-CD28 antibodies (Dynabeads), were added, 1 bead for 10 cells.

### Example 6

In this Example, the effects of T9_{IL-33} cells on target organs of GVHD, which included gut, liver and skin, were analyzed.

Mice underwent allo-HCT. Briefly, Balb/c, C3H.SW recipients received 900, 1100 total body irradiation (¹³⁷Cs source), respectively, on day −1. Recipient mice were injected intravenously with T cell depleted (TCD) bone marrow cells (5×10⁶) plus 1×10⁶ in vitro differentiated T cells (T0,T1,T2,T9 and T9 IL-33) from C57BL/6 with type of ST2−/−, IL-9−/− for Balb/C , 3×10⁶ for C3H.SW at day 0. Mice were housed in sterilized micro-isolator cages and maintained on acidified water (pH <3) for 3 weeks. Survival was monitored daily. Clinical GVHD scores were assessed weekly. According to animal protocols approved by the Institutional Review Board, mice were killed when the clinical score achieved 6.5.

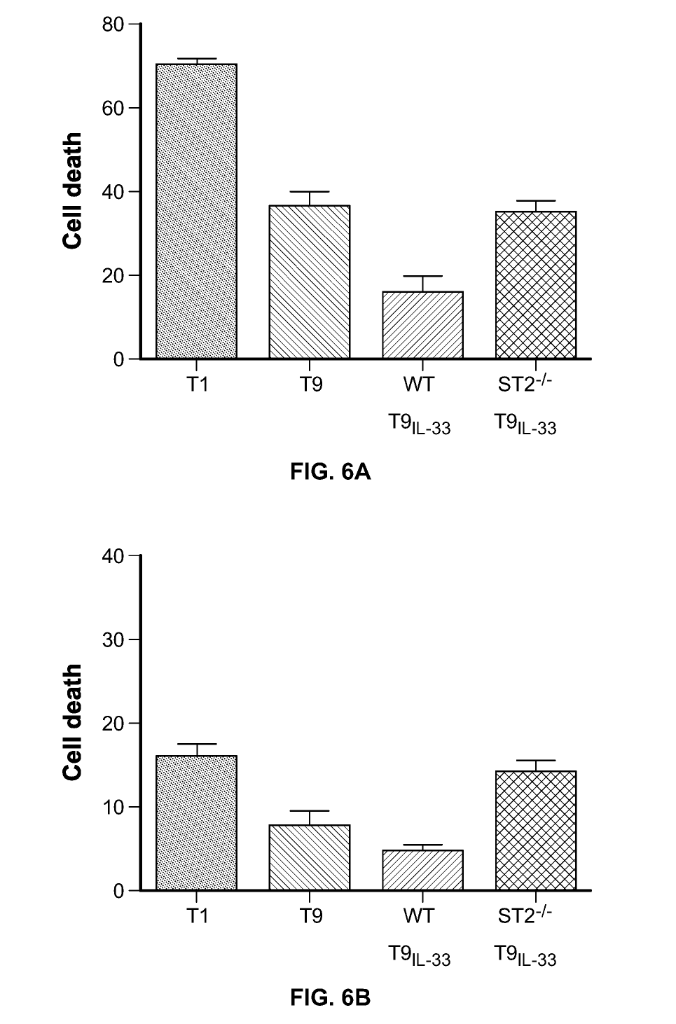

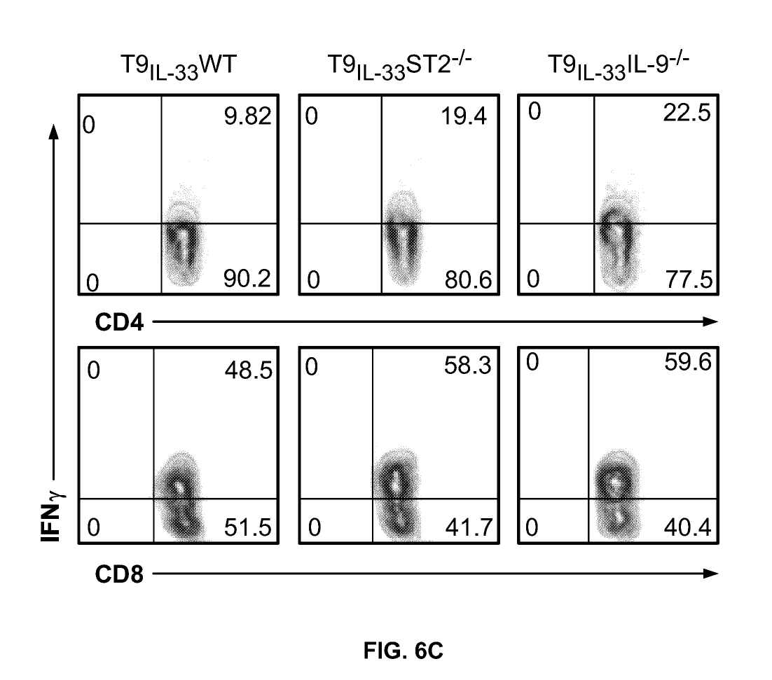

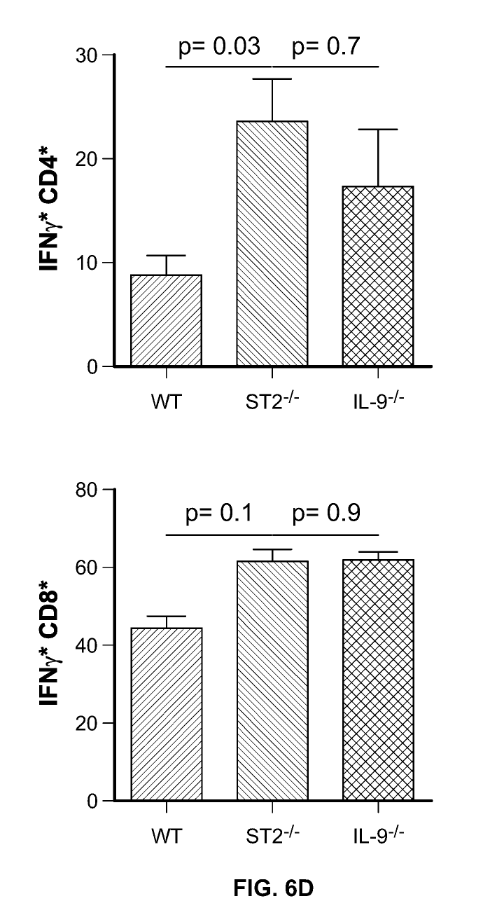

Pathological analysis of target organs of GVHD, which include gut, liver and skin, showed less tissue damage in mice that received T9_{IL-33}, compared to all other groups. Ex vivo analysis of target organs showed a decrease in interferon (IFN)γ-producing T cells, the main driver of GVHD tissue damage when T9_{IL-33} were transferred. This effect was abolished in mice receiving T9_{IL-33} cells derived from ST2−/− or IL-9−/− T cells (FIG. 6C). No difference, however, in frequencies and numbers were detected of regulatory T cells or innate lymphocytes cells (ILCs), which are known to be involved in GVDH protection (data not shown).

Co-culture of allogeneic T9_{IL-}33 cells with primary Balb/C derived colonic epithelial cells (BALB 5047 cells) showed less cell death when compared to allogeneic T1 and T9 cells, whereas high apoptosis induction was reduced when T1 cells separated in transwell culture from epithelial cells (FIGS. 6A & 6B).

### Example 7

In this Example, the effects of T9_{IL-33} cells on the severity and occurrence of GVHD, as well as the effects of T9_{IL-33} cells on GVL, were analyzed.

Balb/C mice were lethally irradiated (900 cGy) one day before bone marrow transplantation. Recipient mice were injected intravenously with 5×10⁶ B6 BM cells and 1×10⁶ enriched in vitro differentiated T cells with either 0.2×10⁶ A20 lymphoma cell line or 2×10⁴ MLL-AF9 cells generated in Balb/C background on day 0. Mice were monitored daily for survival and leukemia development and weekly for GVHD score. Death was attributed to leukemia based on a high percentage of eGFP⁺ cells and death to GVHD only if the mice had a low percentage of eGFP⁺ cells and a GVHD score of 6.5. Cells from peripheral blood, BM, spleen, and liver were analyzed by flow cytometry.

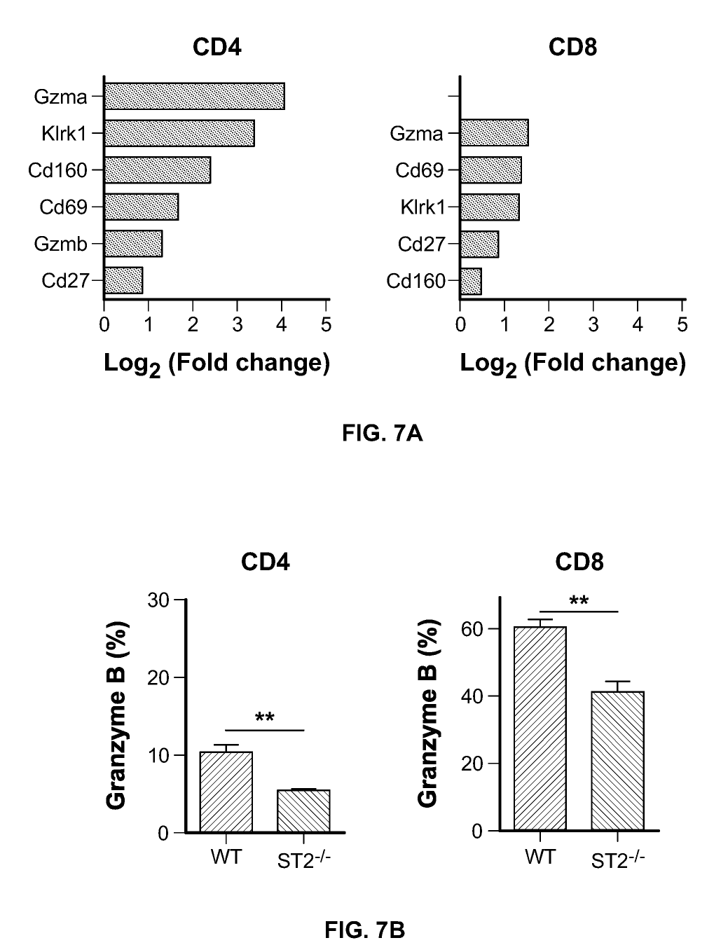

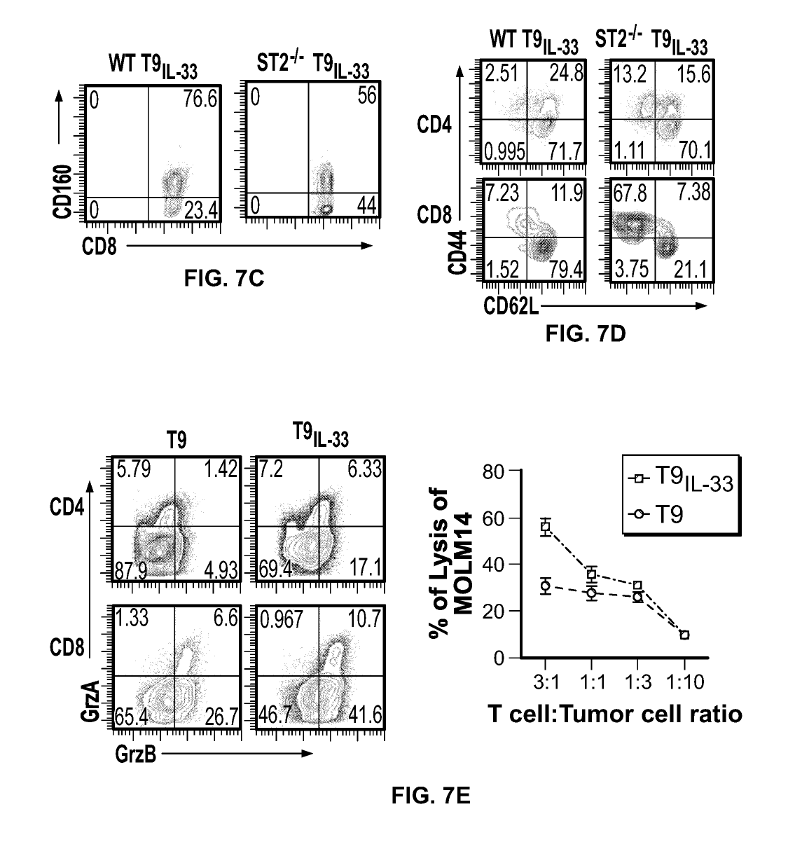

Transcriptome analysis of T9_{IL-33} cells from wild-type and ST2−/− T cells showed upregulation of molecules implicated in anti-leukemic activity (GrazA, GrazB, CD160, KLRK1) and activation marker of central memory (CD69, CD27). Such upregulation was confirmed at the protein level. GrazB, CD160, and T9IL-33 showed higher central memory phenotype in mouse CD62L+ CD27+ (FIG. 7B) and human CD45RO+ CD45RA+ CCR7+ (FIG. 7C), which has been shown to be integral to immunotherapies associated with tumor regression.

Furthermore, T9_{IL-33} cells revealed higher anti-leukemic activity in vitro when cultured with retrovirally transduced MLL-AF9 leukemic cells in cytolytic assays. A low level of cytotoxicity was observed when T9_{IL-33} cells were co-cultured with syngeneic, compared to allogeneic leukemia cells showing a high rate of specify of T9_{IL-33} cells related to minor or major alloantigen reaction (FIG. 7D). Similarly, human T9_{IL-33} cells demonstrated higher in vitro anti-leukemic cytolytic activity when incubated with MOLM14, an AML tumor cell line expressing FLT3/ITD mutations (FIG. 7E).

In vivo GVL experiments with MLL-AF9 induced leukemia, and adoptive transfer of T9_{IL-33} cells resulted in increased survival compared to transfer of T9_{IL-33} cells generated from ST2−/− or IL-9−/− T cells (see Example 8).

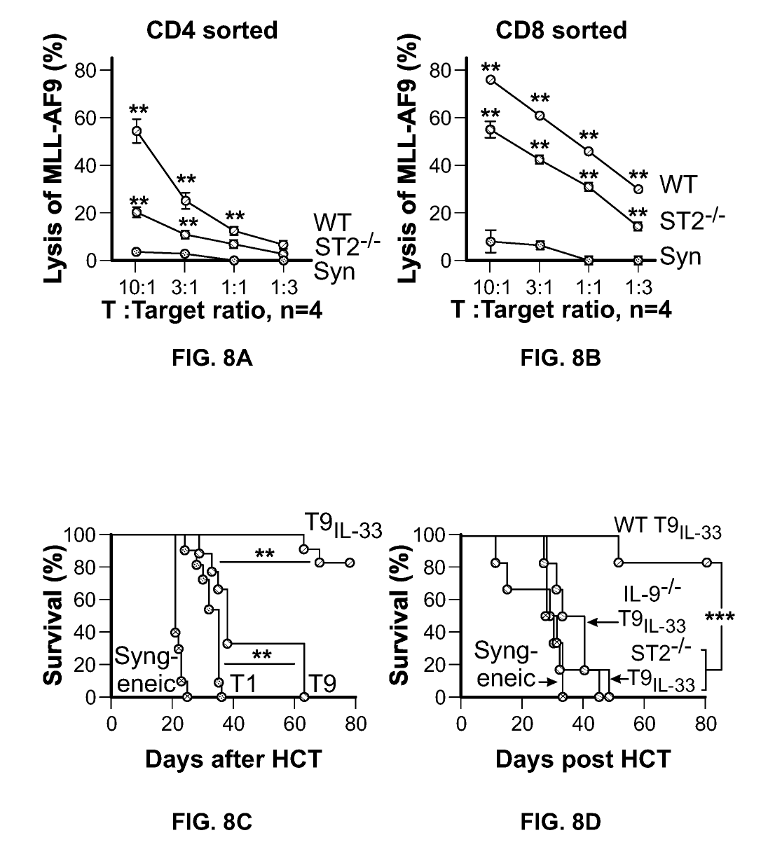

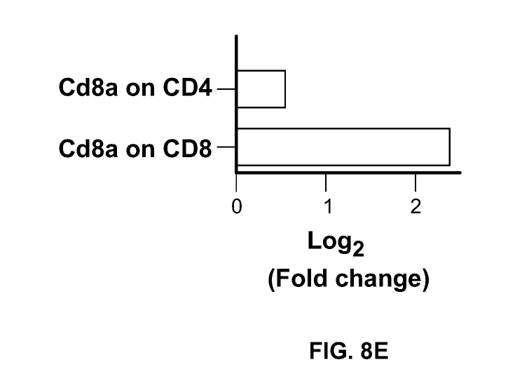

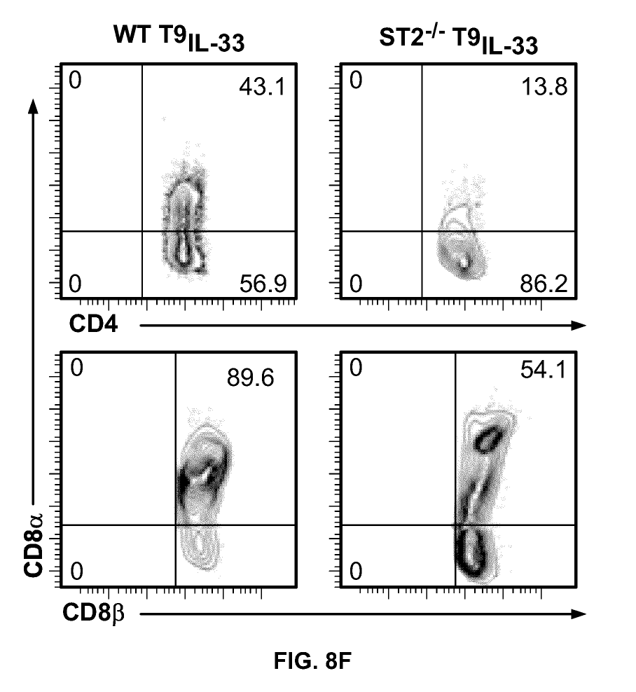

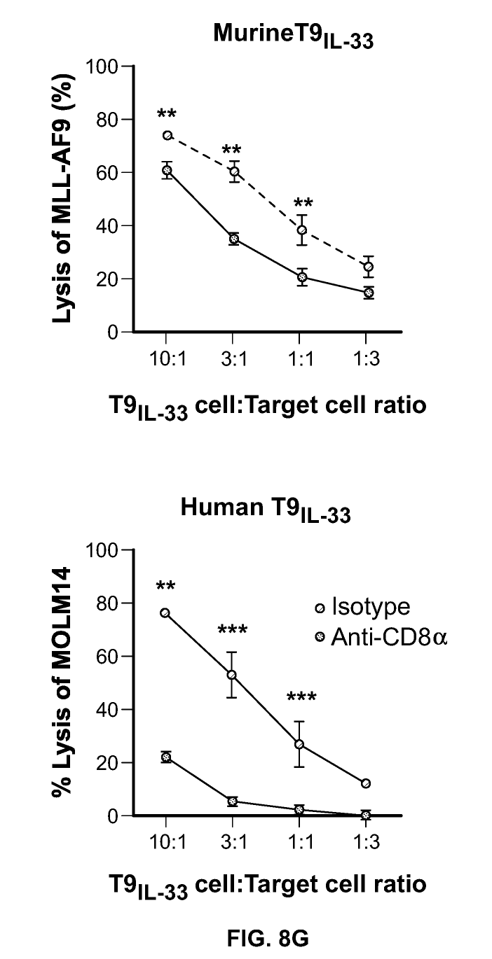

Furthermore, investigations into the possible mechanism of activation using transwell assays revealed that both soluble factors and cell contact between Th9_{IL-33} and Tc9_{IL-33} T cells resulted in maximum killing (FIGS. 8A & 8B). Transcriptome analysis of T9_{IL-33} cells from wild-type and ST2−/− T cells showed upregulation of CD8α. CD8α blockade with neutralizing antibody during human T9_{IL-33} differentiation reduced the cytotoxicity of both murine T9_{IL-33} and human T9_{IL-33}cells (FIG. 8G).

### Example 8

In this Example, the impact of ST2/IL-33 signaling on T9 activity was analyzed. Further, the in vivo function of allogeneic T9_{IL-33} cells in comparison with T0, T1, T2, and T9 cells was analyzed in a histocompatibility antigen mismatch model of HCT.

Materials and Methods

Mice

BALB/c (H-2^{d}), C57BL/6 (B6, H-2^{b}, CD45.2⁺), C57BL/6.Ptprca (B6-SJL, H-2^{b}, CD45.1⁺) and C3H.SW (H-2^{b}, CD45.2⁺) mice were purchased from the Jackson Laboratories. B6 ST2^{-/-}(CD45.2⁺) mice were provided by Dr. Andrew McKenzie from University of Cambridge, UK, and B6 IL-9^{-/-} (CD45.2⁺) mice were provided by Dr. Alexander Rosenkranz from University of Graz, Austria. Animal protocols were approved by the Institutional Animal Care and Use Committee at Indiana University School of Medicine.

T-Cell Differentiation

To investigate the impact of ST2/IL-33 signaling on T9 activity, total T cells were differentiated into T9 cells in the presence (T9_{IL-33}) or absence (T9) of IL-33. Particularly, total CD4+ and CD8+ T cells were purified from spleens via magnetic bead selection (Miltenyi Biotec). These cells were plated at a concentration of 1×10⁶ cells/mL and activated with 1 μg/mL plate-bound anti-CD3 (2C11) and 5-10 μg/mL soluble anti-CD28 (37.51). CD4⁺ and CD8⁺ cells were polarized toward either T0 (without cytokines), T1 (1 ng/mL IL-2 and 20 ng/mL IL-12), T2 (20 ng/mL IL-4), T9 (4 ng/mL TGF-13 and 10 ng/mL IL-4) or T9_{IL-33} (4 ng/mL TGF-13, 10 ng/mL IL-4, and 10 ng/mL IL-33) in Dulbecco's modified Eagle medium (DMEM) supplemented with 10% fetal bovine serum (FBS), 2 mM L-glutamine, 1% penicillin/streptomycin, 1 mM sodium pyruvate, and 50 μM β-mercaptoethanol (Life Technologies). On day 3, the cells were expanded with fresh growth media in the presence of additional cytokines at the same concentrations as in day 0 medium. On day 5, the cells were collected, washed and prepared for phenotypic analysis or adoptive transfer into recipient mice.

Human T cells, CD4⁺ or CD8⁺ T cells were purified from peripheral blood mononuclear cells (PBMCs) of healthy donors and activated with anti-CD3/CD28 microbeads (Life Technologies). Both the CD4⁺ and CD8⁺ cells were polarized toward either T1 (1 ng/mL IL-2 and 20 ng/mL IL-12), T2 (20 ng/mL IL-4), T9 (4 ng/mL TGF-13 and 10 ng/mL IL-4) or T9_{IL-33} (4 ng/mL TGF-13, 10 ng/mL IL-4, and 10 ng/mL IL-33) in complete RPMI medium with 10% human AB serum. On day 3, the cells were expanded with fresh medium in the presence of additional cytokines at the same concentrations as on day 0. On day 7, the cells were collected, washed and prepared for phenotypic analysis and in vitro assays.

Induction and Assessment of GVHD

Mice underwent allo-bone marrow transplantation. Briefly, BALB/c and C3H.SW recipients received 900 and 1100 cGy total body irradiation (137Cs source), respectively, on day −1. Recipient mice were injected intravenously with T cell-depleted bone marrow (TCD BM) cells (5×10⁶) plus in vitro differentiated T0, T1, T2, T9, WT T9_{IL-33}, ST2^{-/-} 9_{IL-33} or IL-9^{-/-} T9_{IL-33} T cells (1×10⁶ for BALB/c, 3×10⁶ for C3H.SW and 3×10⁶ for B6) from either syngeneic or allogeneic donors at day 0. TCD BM cells from donors were prepared with CD90.2 Microbeads (Miltenyi Biotec). Mice were housed in sterilized microisolator cages and maintained on acidified water (pH <3) for 3 weeks. Survival was monitored daily, and clinical GVHD scores were assessed weekly. Mice were euthanized when the clinical scores reached 6.5, in accordance with animal protocols approved by the Institutional Review Board.

Induction and Assessment of GVT Effect

BALB/c or C3H.SW mice were lethally irradiated (900 or 1100 cGy, respectively) on day −1. Recipient mice were injected intravenously with 5×10⁶ syngeneic or allogeneic TCD BM cells and 1×10⁶ for BALB/c and 3×10⁶ for C3H.SW in vitro differentiated syngeneic T9_{IL-33} cells or B6 T0, T1, T9, T9_{IL-33} WT or T9_{IL-33} ST2^{-/-} cells as well as 10⁴ GFP-MLL-AF9 leukemic cells generated from C3H.SW or BALB/c BM as described on day 0. Mice were monitored daily for survival and leukemia development, and GVHD was scored weekly. Death was attributed to leukemia based on a high percentage of eGFP⁺ cells and death to GVHD only if the mice had a low percentage of eGFP⁺ cells and a GVHD score ≥6.5. Cells from peripheral blood, BM, spleen, and liver were analyzed by flow cytometry.

Flow Cytometry

All antibodies (Table 1) and reagents for flow cytometry were purchased from eBioscience, unless stated otherwise. The cells were pre-incubated with purified anti-mouse CD16/CD32 mAb for 15 minutes at 4° C. to prevent nonspecific binding of the antibodies. The cells were subsequently incubated for 30 minutes at 4° C. with antibodies for surface staining. Fixable viability dye (FVD) was used to distinguish live cells from dead cells. The FoxP3/Transcription Factor Staining Buffer Set and the Fixation and Permeabilization Kit were used for intracellular staining. For cytokine staining, cells were re-stimulated with anti-CD3 (10 μg/ml) for 4-6 hours, and brefeldin A was added for the last 2 hours of culture.

TABLE 1
Flow Antibody List

| | | | |
|---|---|---|---|
| Antibody | Company | Clone | Fluorochrome |

Mouse

| | | | |
|---|---|---|---|
| CD4 | eBioscience | GK1.5 | PE/PerCP- |
| | | | eFluor ® 710 |
| CD8α | eBioscience | 53-6.7 | FITC/PE-CY7 |
| CD8β | eBioscience | eBioH35-17.2 | PE |
| | | (H35-17.2) | |
| IFNγ | eBioscience | XMG1.2 | PE/APC/PerCP- |
| | | | Cy5.5 |
| IL-9 | eBioscience | RM9A4 | APC |
| Perforin | eBioscience | eBioOMAK-D | APC |
| Granzyme B | eBioscience | NGZB | PE |
| CD27 | eBioscience | LG.7F9 | PE |
| CD44 | eBioscience | IM7 | FITC/eFluor ® 450 |
| CD62L | eBioscience | MEL-14 | PE |
| KLRG1 | eBioscience | 2F1 | APC |
| CD160 | eBioscience | eBioCNX46-3 | APC |
| | | (CNX46-3) | |
| CD90 | eBioscience | 30-H12 | FITC/PE-CY7 |
| AREG | R&D | Polyclonal | Unconjugated |
| HVEM | eBioscience | LH1 | APC |
| Foxp3 | eBioscience | FJK-16s | PE-CY7 |
| ST2 | eBioscience | RMST2-2 | APC/PerCP- |
| | | | eFluor ® 710 |
| ST2 | mdbioproduct | Dj8 | PE |
| PU.1 | Santa Cruz | B-9 | Unconjugated |
| Ki67 | eBioscience | SolA15 | PerCP-eFluor ® |
| | | | 710/eFluor ® 450 |
| α4β7 | eBioscience | DATK32 (DATK- | APC |
| | | 32) | |
| CRK | Thermo Fishers | 4G11E8 | Unconjugated |
| CCR5 | eBioscience | HM-CCR5 (7A4) | PerCP-Cy5.5 |
| Lgr5 | R&D | 889901 | APC |
| EpCam | eBioscience | G8.8 | PE-CY7 |

Human

| | | | |
|---|---|---|---|
| CD4 | eBioscience | OKT4 | PE/PerCP- |
| | | | eFluor ® 710 |
| CD8 | eBioscience | OKT8 | FITC/APC |
| IFNγ | eBioscience | 4S.B3 | PE |
| IL-9 | eBioscience | MH9D1 | APC/PerCP- |
| | | | eFluor ® 710 |
| Granzyme B | eBioscience | GB11 | PE |
| Granzyme K | eBioscience | G3H69 | PerCP-eFluor ® |
| | | | 710 |
| AREG | R&D | Polyclonal | Unconjugated |
| ST2 | mdbioproduct | B4E6 | FITC |

Mixed Lymphocyte Reaction (MLR)

Purified splenic total T cells from B6 WT or ST2^{-/-} mice were cultured with allogenic T cell-depleted and irradiated splenocytes (3000 cGy) from BALB/c or C3H.SW mice in the presence of polarizing cytokines (10 ng/ml IL-12 for T1 and 4 ng/mL TGF-β, 10 ng/mL IL-4 and 10 ng/mL for IL-33 T9_{IL-33}). On day 3, the cells were expanded with fresh growth media in the presence of additional cytokines at the same concentrations as in day 0 medium. Splenic T cells from BALB/c or C3H.SW mice cultured under the same conditions were used as syngeneic controls.

In Vitro Cytotoxicity Assay

Purified splenic T cells were primed in a MLR in the presence of polarizing cytokines (IL-4, transforming growth factor β and IL-33) for 5 days. Total T9_{IL-33} cells or sorted CD4 and CD8 from B6 WT T9_{IL-33}, ST2^{-/-} T9_{IL-33} or C3H.SW T9_{IL-33} cultures were incubated with C3H.SW GFP-MLL-AF9 leukemic cells at different ratios. After 6 hours, cells were washed, stained with viability dye and analyzed by flow cytometry. Human T9 or T9_{IL-33} were labelled with 5 μM CFSE and co-incubated with MOLM14 leukemic cells labelled with 0.5 μM CFSE (Life Technologies). After 6 hours, cells were washed and analyzed by flow cytometry. For imaging, cells were labelled with CD8α, CD8β and SYTOX (SYTOX was added 5 minutes before acquisition), and images were acquired using Image Stream (Amnis) after 3 hours of co-incubation.

Colonic Epithelial Cell Apoptosis Assay

A BALB/c primary colonic epithelial cell line (BALB-5047 Cell Biologic) was co-cultured together or separately through a Transwell with in vitro differentiated T9, WT T9_{IL-33} or ST2^{-/-} T9_{IL-33} cells at a ratio of 1:1 in with anti-IL-20Rb, anti-AREG, anti-HVEM (from R&D Systems) or the appropriate isotype control. Six hours later, cells were washed, stained with FVD and analyzed by flow cytometry.

Human T1, T9 or T9_{IL-33} cells were co-cultured with the primary colonic epithelial cell line (HNNC) in the presence of anti-human AREG or isotype control in the same conditions as described above.

Isolation of Intestinal Cells

Single-cell suspensions were prepared from intestines. Briefly, intestines were flushed with phosphate-buffered saline (PBS) to remove fecal matter and mucus. Fragments (<0.5 cm) of intestines were digested in 10 ml DMEM containing collagenase type B (2 mg/ml; Roche), deoxyribonuclease I (10 pg/ml; Roche), and 4% bovine serum albumin (Sigma-Aldrich) at 37° C. with shaking for 90 minutes. The digested mixture was then diluted with 30 ml plain DMEM, filtered through a 70-pm strainer and centrifuged at 850 g for 10 minutes. The cell pellets were suspended in 5 ml of 80% Percoll (GE Healthcare), overlaid with 8 ml of 40% Percoll and spun at 2000 rpm for 20 minutes at 4° C. without braking. Enriched lymphocytes were collected from the interface.

CD8α Blocking

Anti-CD8a blocking antibody for mouse (53-6.7) or human (LT8) was added (both at 50 μg/ml) during differentiation of T9IL-33 cells or during co-incubation with MLL-AF9 cells.

Cell Sorting

CD4⁺ or CD8⁺ T cells were harvested from in vitro differentiated WT T9_{IL-33} or ST2^{-/-} T9_{IL-33} cells for quantitative reverse transcription polymerase chain reaction (qPCR) and NanoString analysis. CD4⁺ T cells and CD8⁺ T cells were sorted from single-cell suspensions of intestine from GVHD mice at day 14 after transplantation of allogeneic T1, WT T9_{IL-33} or ST2^{-/-} T9_{IL-33} cells for qPCR. Cell sorting was performed using a BD FACSAria (BD Bioscience).

qPCR

Total RNA was isolated from sorted cells using the RNeasy Plus Mini Kit (QIAGEN). cDNA was prepared with SuperScript® VILOTM cDNA Synthesis Kit (Invitrogen). qPCR was performed using SYBR Green PCR mix on an ABI Prism 7500HT (Applied Biosystems). Thermocycler conditions included 2-minute incubation at 50° C., then 95° C. for 10 minutes; this was followed by a 2-step PCR program of 95° C. for 5 seconds and 60° C. for 60 seconds for 40 cycles. β-Actin was used as an internal control to normalize for differences in the amount of total cDNA in each sample. The primer sequences were as follows:

Actin forward:
(SEQ ID NO: 1)
5′-CTCTGGCTCCTAGCACCATGAAGA-3′
Actin reverse:
(SEQ ID NO: 2)
5′-GTAAAACGCAGCTCAGTAACAGTCCG-3′
ST2L forward:
(SEQ ID NO: 3)
5′-AAGGCACACCATAAGGCTGA-3
ST2L reverse:
(SEQ ID NO: 4)
5′-TCGTAGAGCTTGCCATCGTT-3′
IL-9r forward:
(SEQ ID NO: 5)
5′-CAC AAA TGC ACC TTC TGG GAC A 3′
IL-9r reverse:
(SEQ ID NO: 6)
5′-TCA CTC CAA CGA TAC GGT CCT T-3′
AREG forward:
(SEQ ID NO: 7)
5′-GGACAATGCAGGGTAAAAGTTGA-3′
AREG reverse:
(SEQ ID NO: 8)
5′-TGAAAGAAGGACCAATGTCATTTC-3′
EGFR forward:
(SEQ ID NO: 9)
5′-TTGGCCTATTCATGCGAAGAC-3′
EFGR reverse:
(SEQ ID NO: 10)
5′:GAGGTTCCACGAGCTCTCTCTCT-3′

NanoString

Sorted CD4⁺ or CD8⁺ T cells from WT or ST2^{-/-} T9_{IL-33} cells were prepared for NanoString analysis. Briefly, cells were lysed in RTL buffer (QIAGEN) on ice. The cell concentration for lysis was 1×10⁴ cells/μL with a total of 5 μL RTL buffer. Lysis samples were frozen in liquid nitrogen immediately and then stored at −80° C. or on dry ice. NanoString analysis was performed with the nCounter® Analysis System at NanoString Technologies. The nCounter® Mouse Immunology Kit, which includes 561 immunology-related mouse genes, was used.

Enzyme-Linked Immunosorbent Assay (ELISA)

Concentrations of IFN-γ, IL-9 and IL-4 in the culture supernatant were measured with the DuoSet ELISA Kits (R&D Systems).

Statistical Analysis

Log-rank test was used for survival analysis. Differences between two groups were compared using unpaired t tests, and differences between three or more groups were compared using one-way analysis of variance followed by Dunnett's multiple comparisons test using GraphPad Prism software, version 6.05. Data in graphs represent mean±SEM. P values less than 0.05 were considered significant.

Results

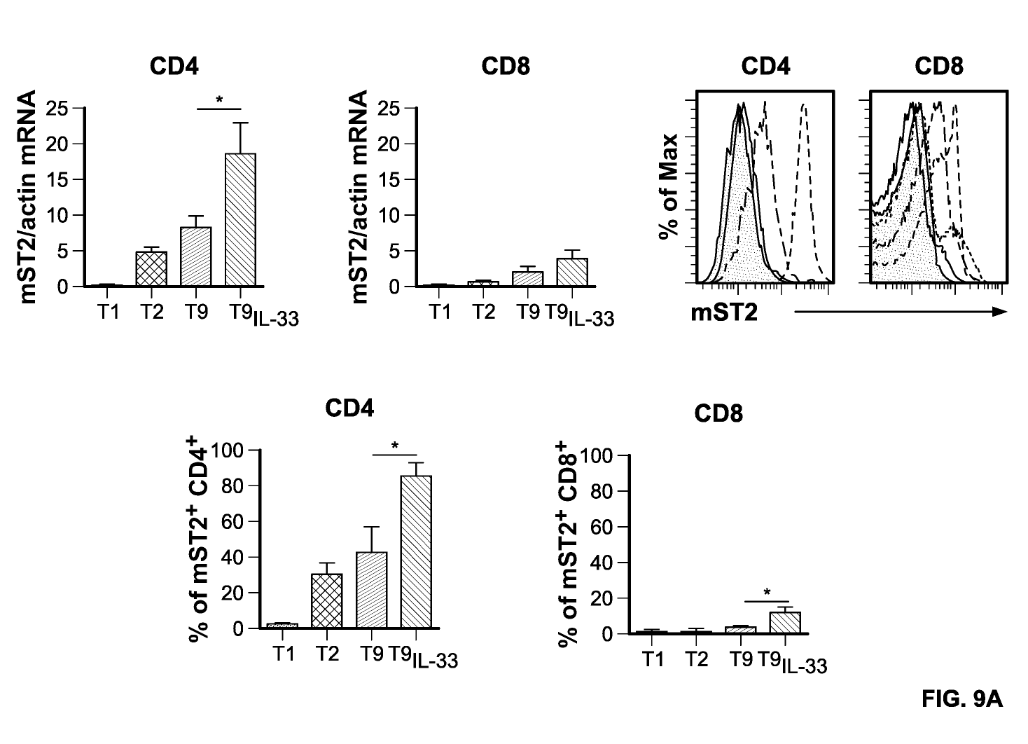

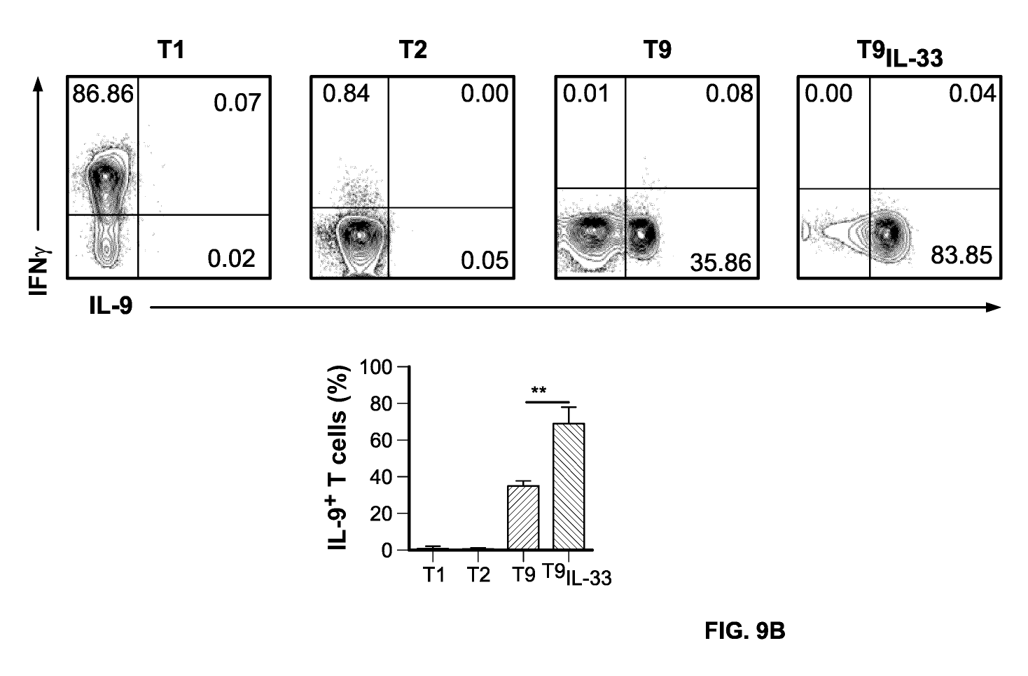

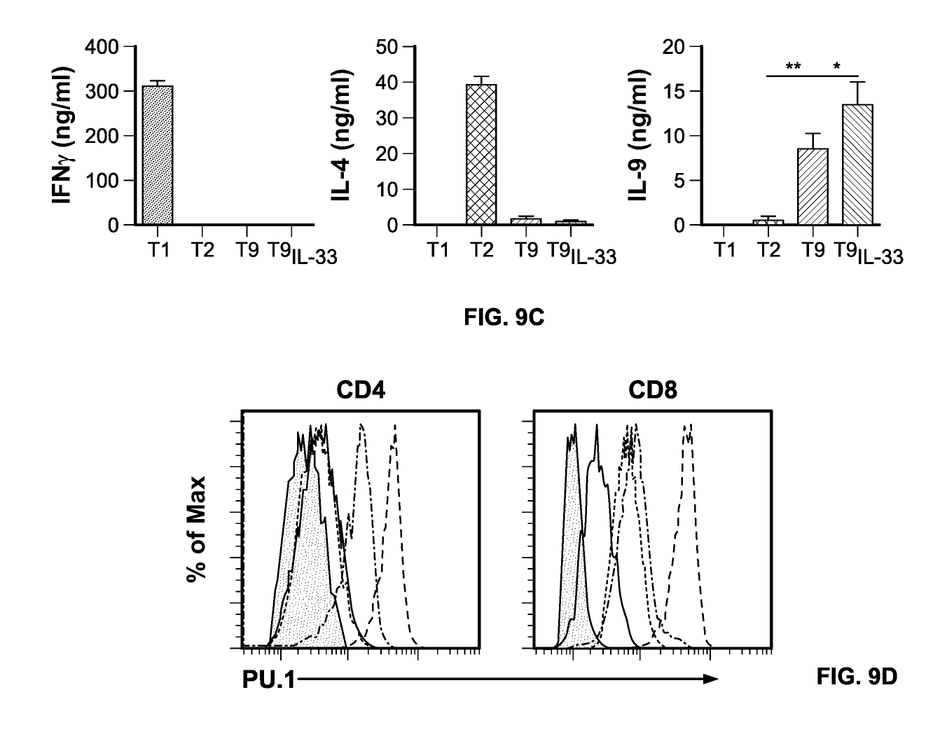

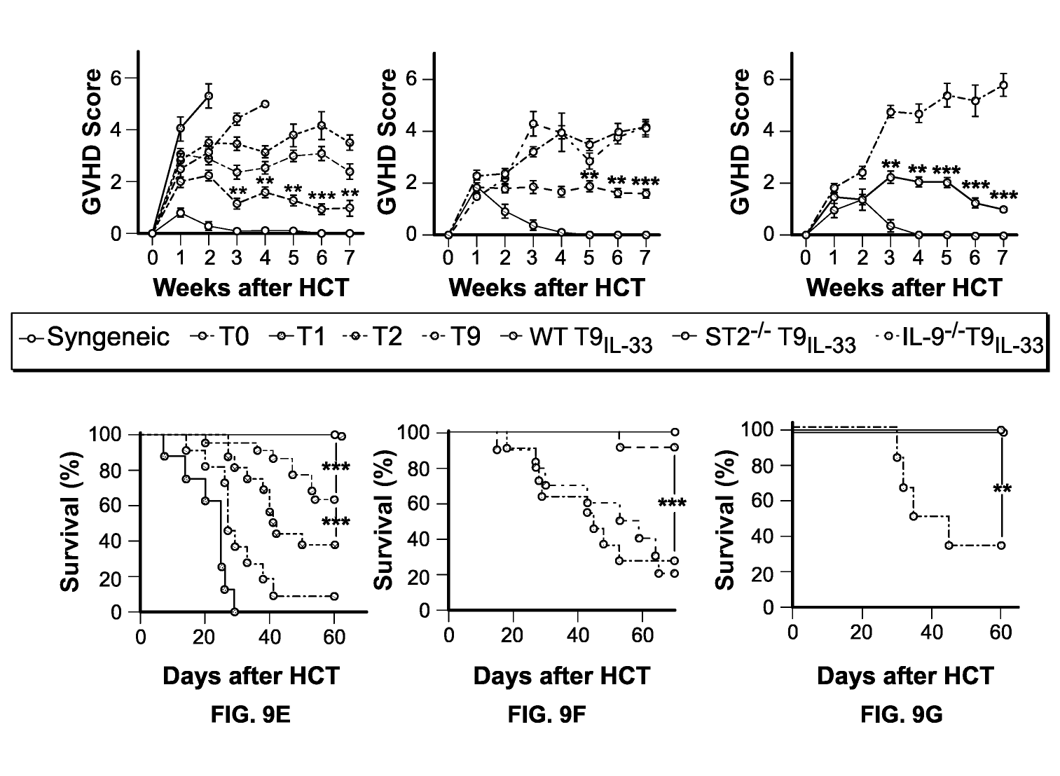

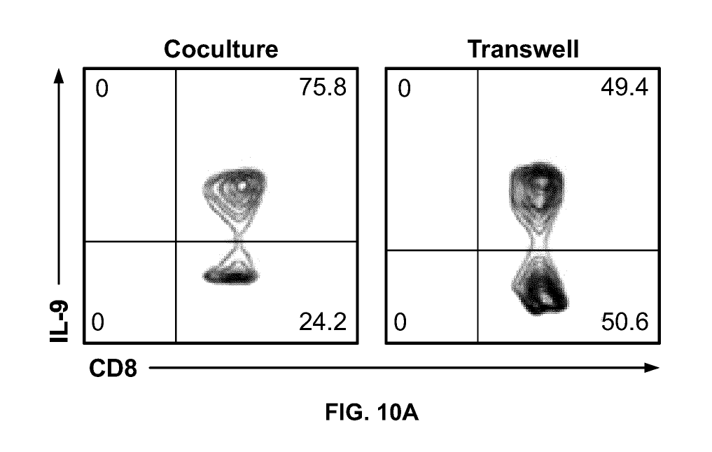

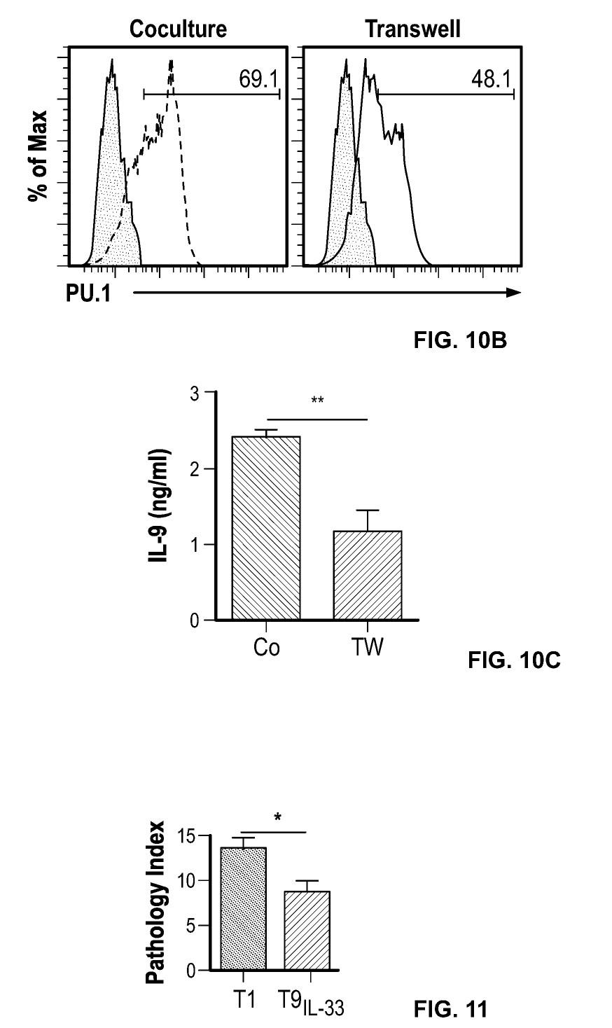

T9 cells expressed mST2 at the transcriptional and protein levels, and mST2 protein expression on T9_{IL-33} cells was further increased on both CD4 (>80% of total CD4 T cells) and CD8 T cells (FIG. 9A). Addition of IL-33 during T9 differentiation also increased IL-9 expression and secretion without inducing expression of interferon (IFN)-γ or IL-4 (FIGS. 9B & 9C), and PU.1, a master transcription factor that promotes IL-9 production, was upregulated in both CD4 and CD8 T cells (FIG. 9D). IL-9 and PU.1 expression, as well as IL-9 secretion by Tc9_{IL-33} cells, were reduced when CD4 and CD8 T cells were separated in Transwell plates during T9_{IL-33} differentiation (FIGS. 10A-10C).

The in vivo function of allogeneic T9IL-33 cells were then evaluated in comparison with T0, T1, T2, and T9 cells in a major histocompatibility antigen mismatch model of HCT. Mice receiving T1 or T0 cells showed severe GVHD and high mortality, whereas mice receiving T2 or T9 cells showed moderate GVHD with 40%-60% survival. Importantly, GVHD was almost completely abrogated in animals receiving T9_{IL-33} cells, with 100% survival in these mice (FIG. 9E). Compared to the WT T9_{IL-33} group, adoptive transfer of T9_{IL-33} cells generated from ST2^{-/-} or IL-9^{-/-} T cells resulted in significantly more severe GVHD and reduced survival (FIG. 9F), indicating that ST2/IL-33 and IL-9 signaling are critical for T9_{IL-33} cell-mediated protection against GVHD. The protective role of the ST2/IL-33 axis was confirmed in a minor histocompatibility antigen (miHA) model of GVHD (FIG. 9G).

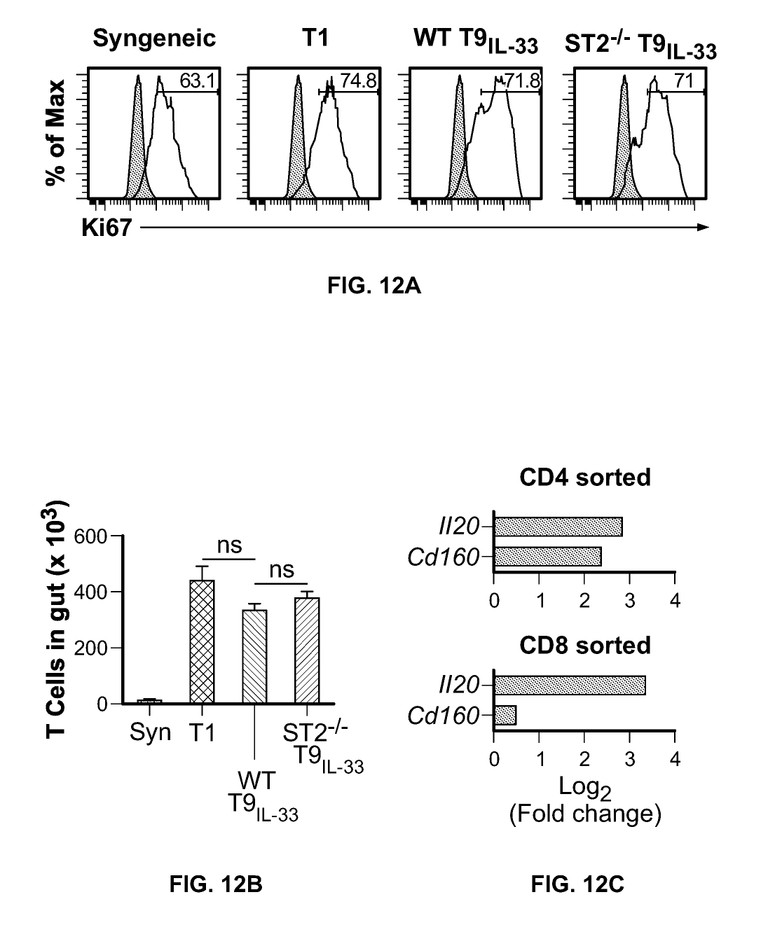

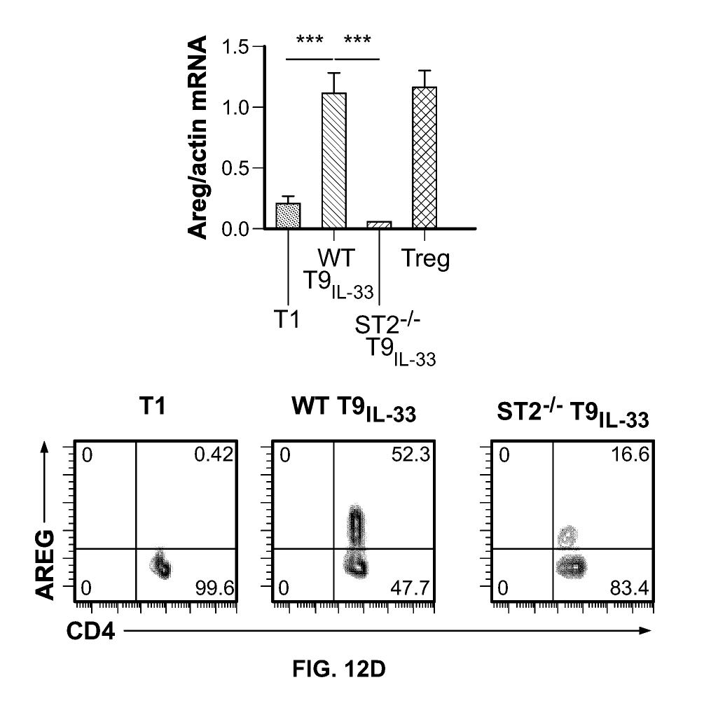

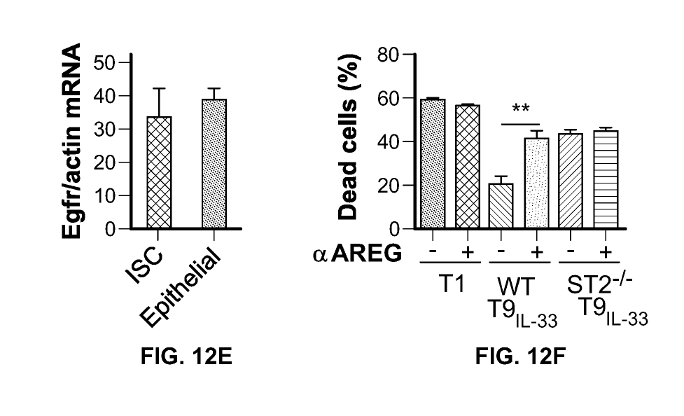

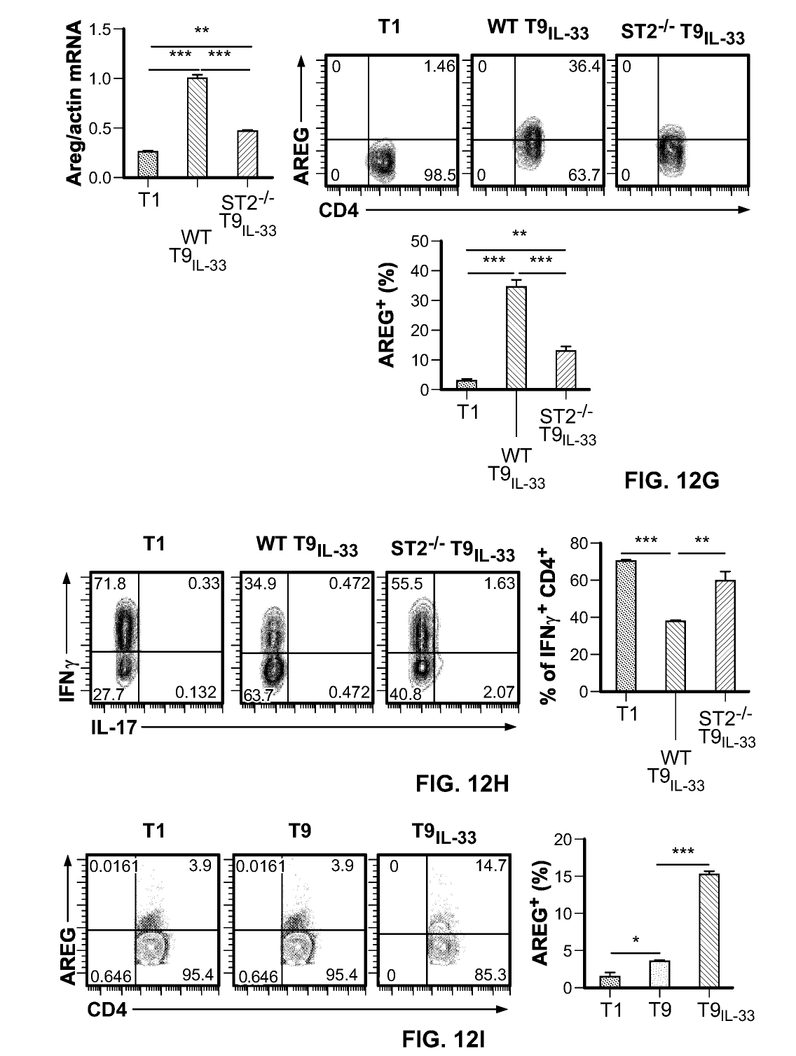

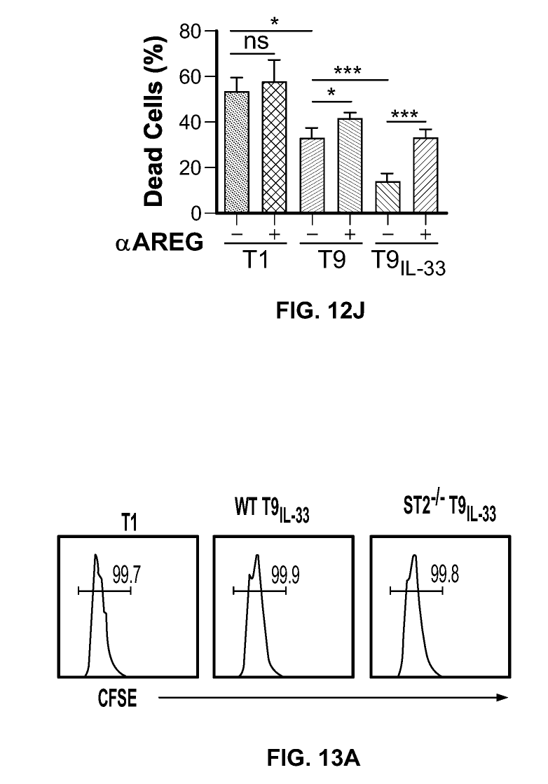

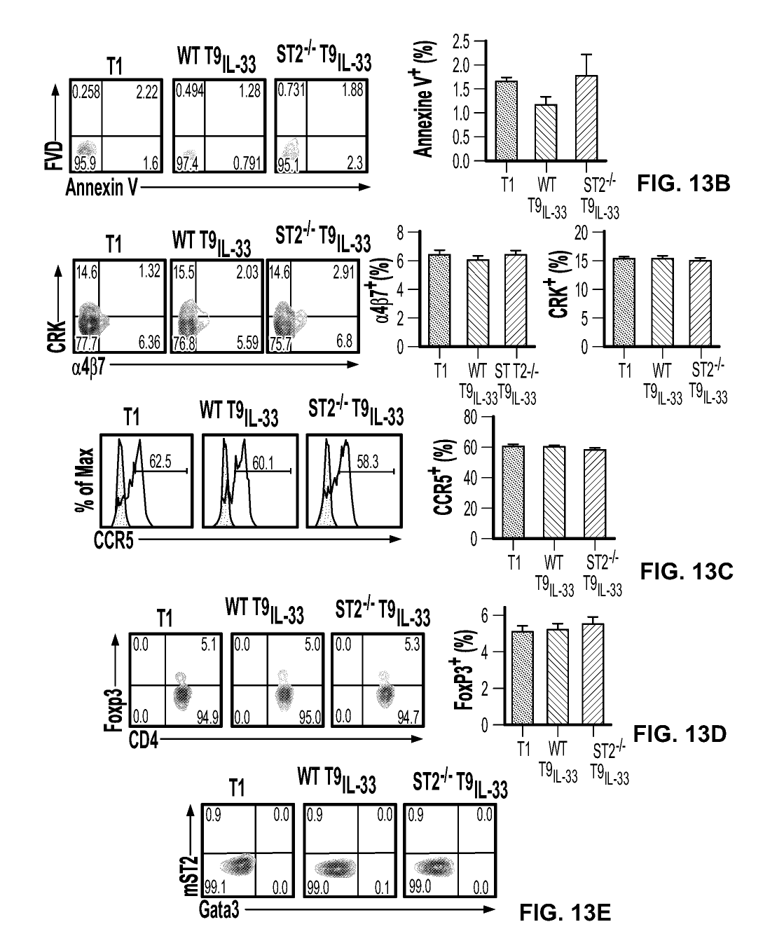

Pathologic examination of the intestines during GVHD showed less tissue damage in mice that received T9IL-33 cells versus T1 cells (FIG. 11). To understand the mechanism(s) responsible for intestinal epithelium protection by T9_{IL-33} cells, several possibilities were explored. First, ex-vivo analysis of T cells in the gut, the major GVHD target organ, showed no difference in T-cell proliferation between groups as measured by Ki67 and carboxyfluorescein succinimidyl ester (CFSE) staining (FIGS. 12A and 13A) or in the total number of gut-infiltrating T cells (FIG. 12B).

Second, possible differences in apoptosis and migration capacities were avoided between WT T9_{IL-33} and ST2^{-/-}T9_{IL-33} cells in the intestine by measuring Annexin-V, α4β7, CRK, and CCR5 expression (FIGS. 13B & 13C).

Third, although mST2 regulatory T cells (T_{regs}) and innate lymphoid cells type 2 (ILC2) reduce GVHD severity, no difference in Treg frequency was observed after transfer of WT T9_{IL-33}, T1 or ST2^{-/-}T9_{IL-33} cells, and ILC2 cells were absent in the intestine of all HCT groups (FIGS. 13D & 13E).

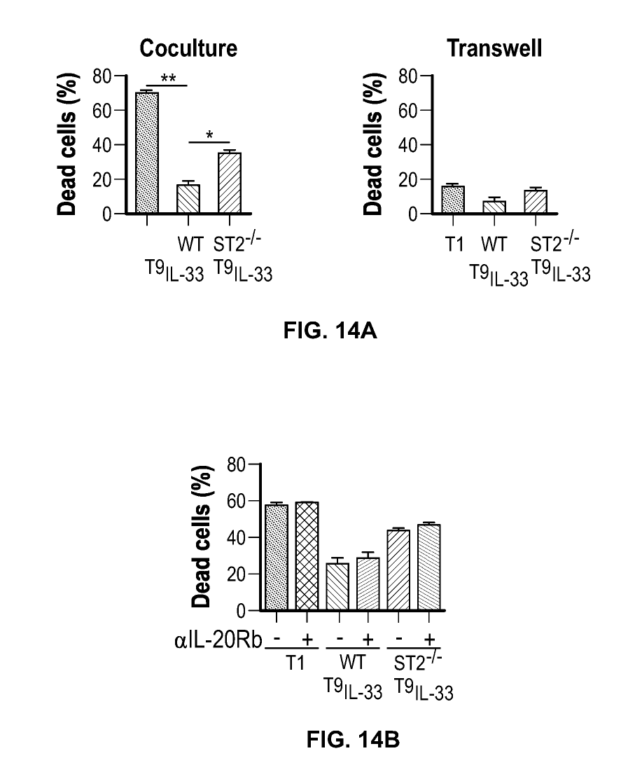

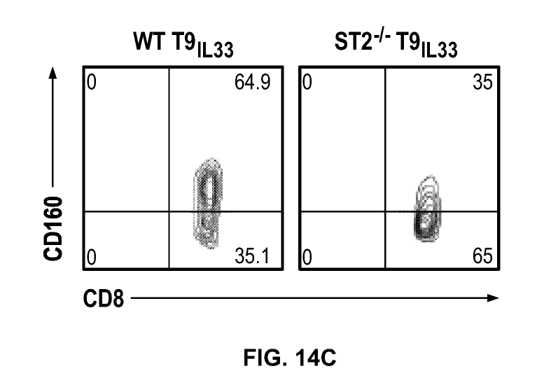

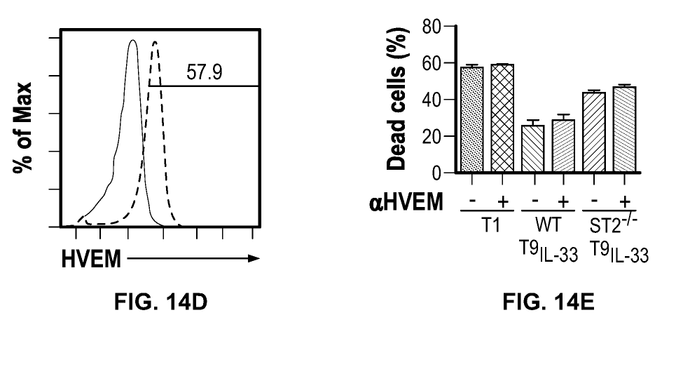

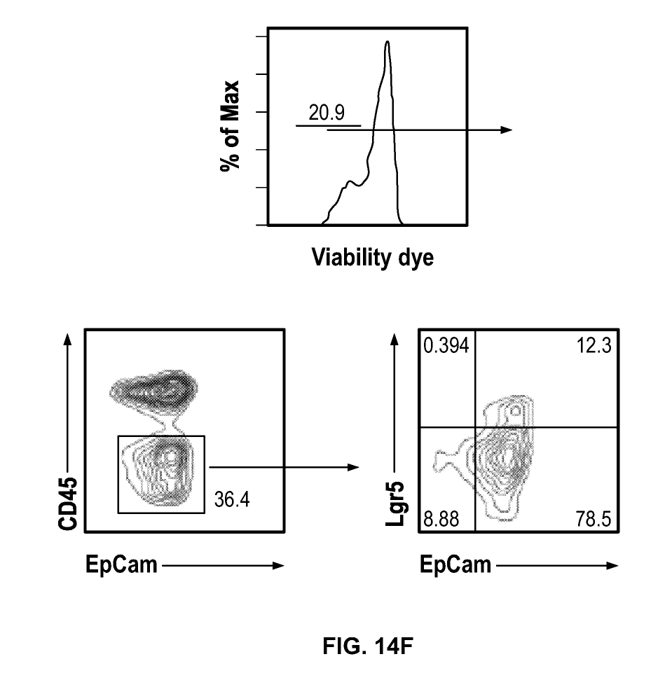

Fourth, Transwell assays of T cells with allogeneic colonic epithelial cells showed that WT T9_{IL-33} cells caused less contact-dependent death of epithelial cells than T1 or ST2^{-/-}T9_{IL-33} cells (FIG. 14A). Next, transcriptome analysis of WT T9_{IL-33} versus ST2^{-/-}T9_{IL-33} sorted CD4 and CD8 T cells showed upregulation of Il20 and Cd160 (FIG. 12C). IL-20Rb blockade did not affect survival of epithelial cells co-cultured with T1, WT T9_{IL-33} or ST2^{-/-}T9_{IL-33} cells (FIG. 14B). CD160 expression was upregulated on WT Tc9_{IL-33}CD8 cells as compared to ST2^{-/-}Tc9_{IL-33}, and its ligand herpes virus entry mediator (HVEM) was expressed on colonic epithelial cells (FIGS. 14C & 14D). Because CD160/HVEM signaling protects mucosa, the CD160 ligand for HVEM was blocked during co-culture of allogeneic T cells with epithelial cells, but this had no impact on epithelial survival (FIG. 14E).

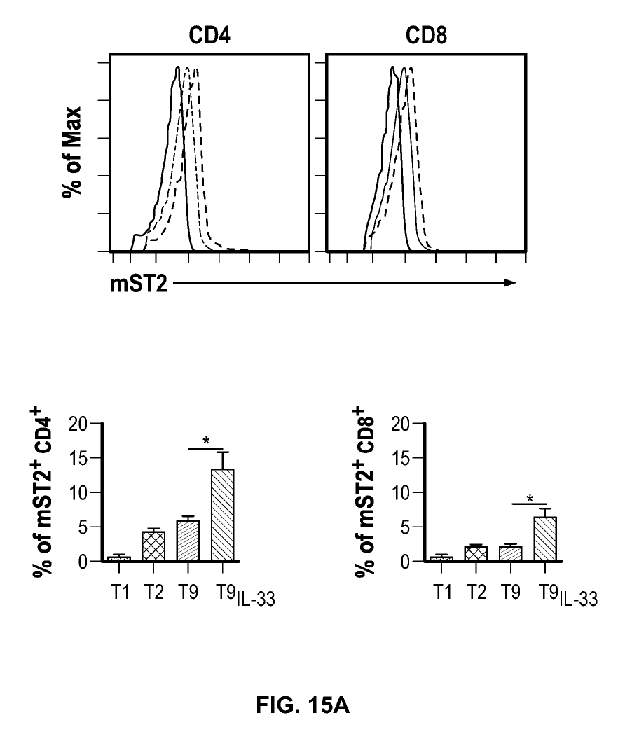

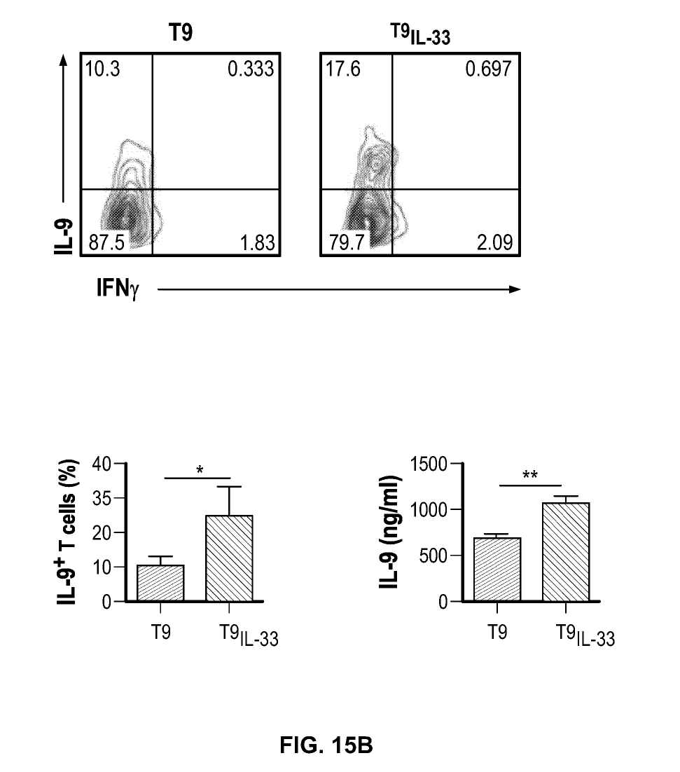

Fifth, to further examine the possible protective mechanism of T9_{IL-33} cells on intestinal mucosa, AREG was explored, because its expression on mST2-expressing cells is involved in tissue repair. AREG expression in T9_{IL-33} cells was greater than that in Ti and ST2^{-/-} T9_{IL-33} cells and similar to that in Tregs (FIG. 12D). In addition, both intestinal epithelial cells and intestinal stem cells (ISCs), the primary target of allogeneic donor T cells during GVHD, expressed epidermal growth factor receptor (EGFR) (FIG. 12E). ISCs and epithelial cells (EpCam+ Lgr5+ and EpCam+ Lgr5−, respectively) were sorted from intestines of naive BALB/c mice (FIG. 14F). This is the first demonstration of EGFR expression in mammalian ISCs, although inactivation of EGFR inhibits ISC growth and division in Drosophila. Blocking AREG in co-cultured epithelial cells and allogeneic WT T9_{IL-33} cells increased their death at rates comparable to those observed with T1 or ST2^{-/-}T9_{IL-33} cells (FIG. 12F), suggesting that T9_{IL-33} cells protect intestinal epithelial cells from the allogeneic response predominantly through AREG binding to EGFR. Ex-vivo analysis of sorted T cells from intestine of HCT models showed that AREG expression was greater in WT T9_{IL-33} cells than in T1 or ST2^{-/-}T9_{IL-33} cells (FIG. 12G), which correlated with a lower frequency of pathogenic cells producing IFN-γ and IL-17 compared with that in T1 or ST2^{-/-}T9_{IL-33} (FIG. 12H). Human T9 cells are poorly characterized. The results demonstrate that differentiation of human T9 cells in the presence of IL-33 enhanced mST2 expression and IL-9 expression/secretion by CD4 and CD8 T cells as compared with other T-cell subsets, including T9 cells, similar to murine T9_{IL-33} cells (FIGS. 15A & 15B). Human T9_{IL-33} cells also exhibited higher AREG expression than T1 and T9 cells (FIG. 12I) and AREG blockade during co-culture with human colonic epithelial cells abolished the protective effect of human T9_{IL-33} cells as well as diminished that for human T9 cells (FIG. 12J). Together, the data suggest that AREG expressed on both human and murine T9_{IL-33} cells provides a strong protection against tissue damage and represents a potential mechanism for the low degree of GVHD observed with adoptive transfer of T9_{IL-33} cells.

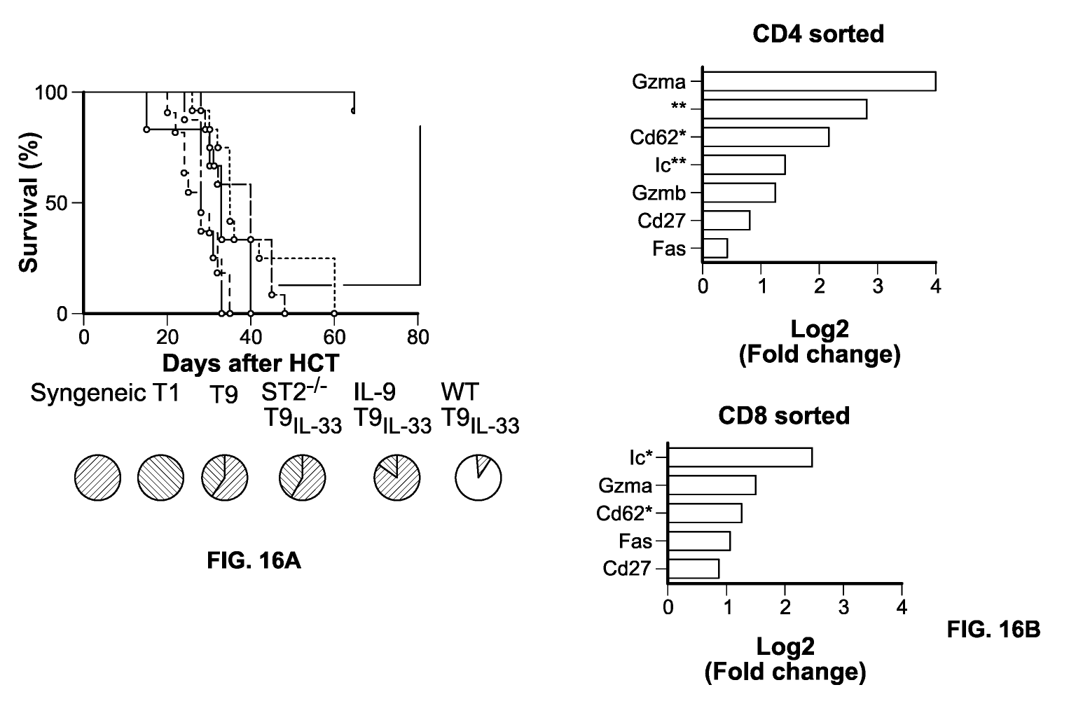

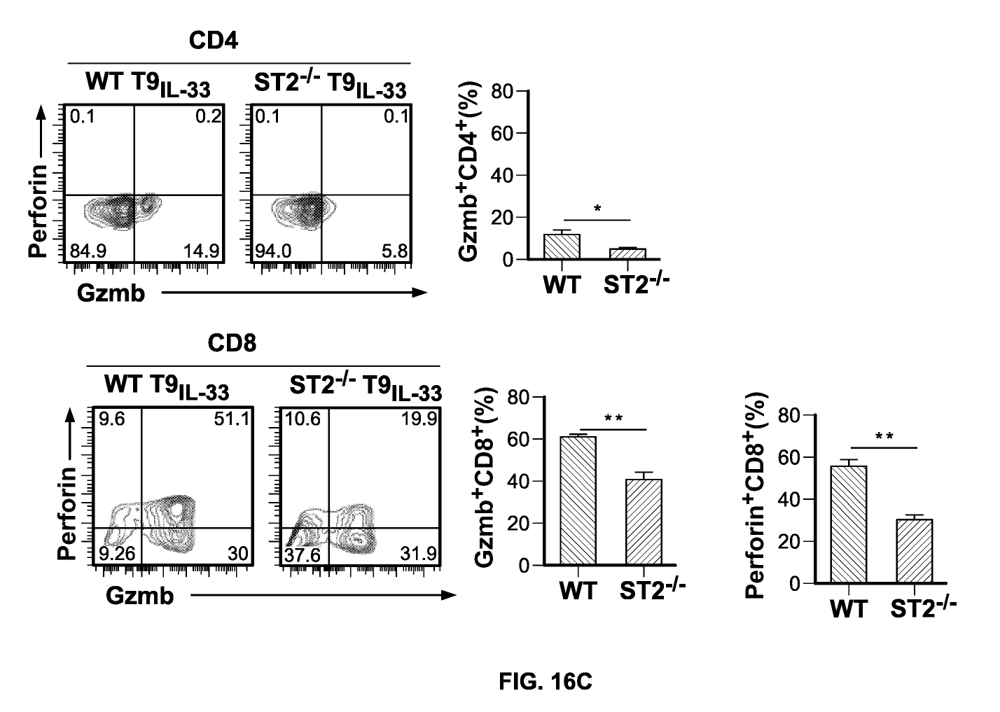

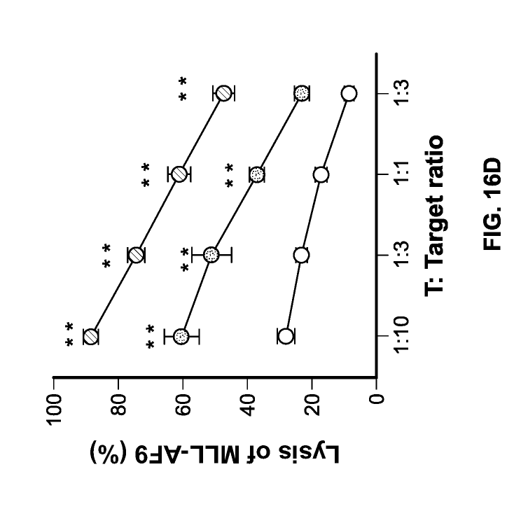

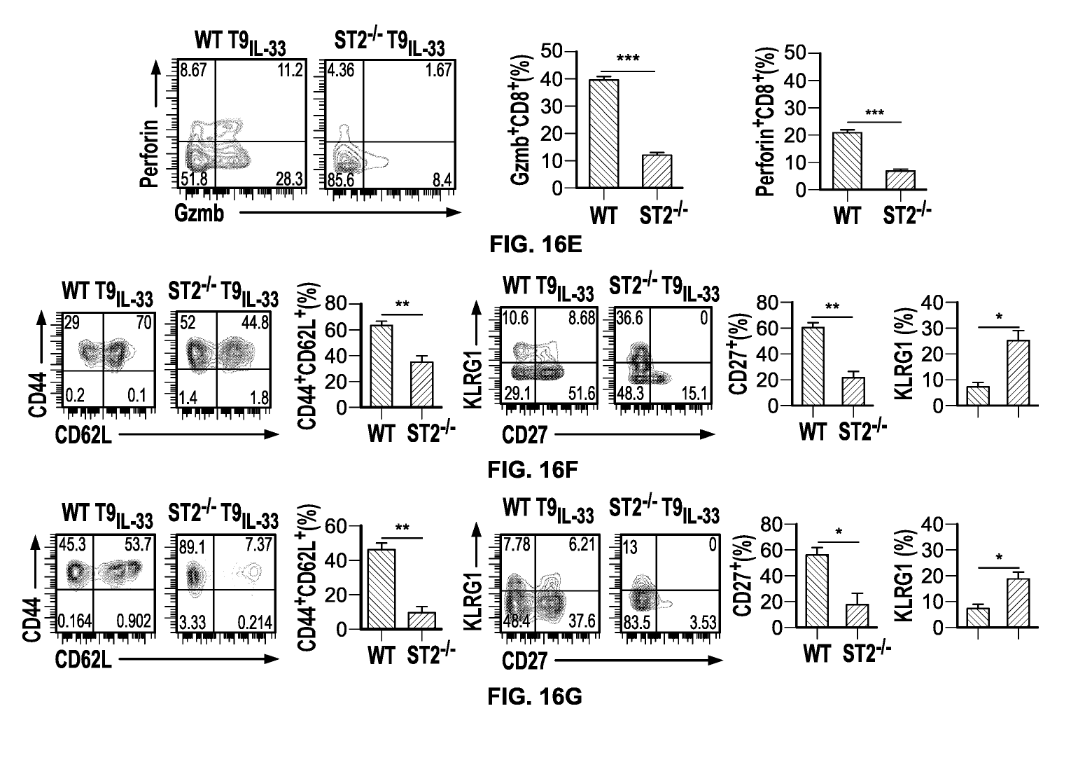

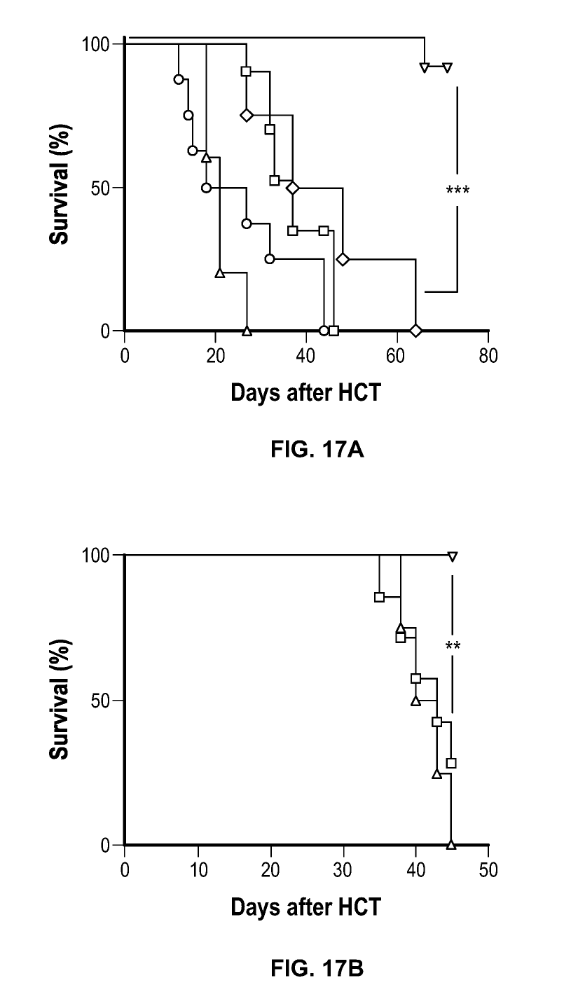

The effects of adoptively transferred T9 IL-33 cells on anti-tumor activity were then investigated. In mice with MLL-AF9 leukemic cells, compared with transfer of syngeneic or allogeneic T cell subsets, transfer of WT T9IL-33 cells resulted in milder GVHD and higher anti-tumor activity, as more than 85% of these mice survived past 80 days post-HCT and were GVHD/tumor-free (FIG. 16A). In contrast, mice receiving T1 cells died early of GVHD. In mice receiving T9 or ST2^{-/-}T9_{IL-33} cells, GVHD onset was delayed compared with that in mice receiving T1 cells, but they all died of leukemia by day 60 (FIG. 16A). A majority of mice receiving IL-9^{-/-}T9_{IL-33} cells died of leukemia (FIG. 16A). The same trends were observed in recipients with lymphoma cell line A20 and in the miHA HCT model (FIGS. 17A & 17B).

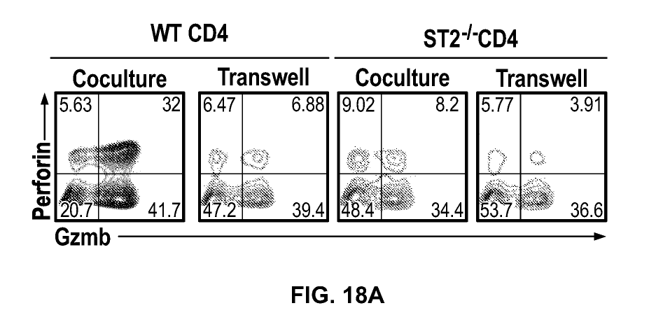

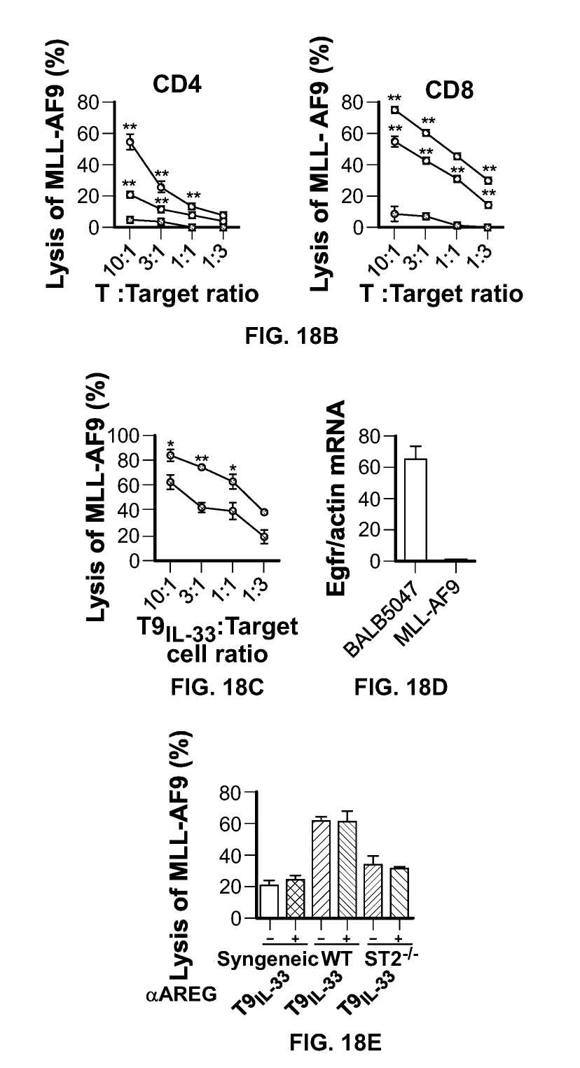

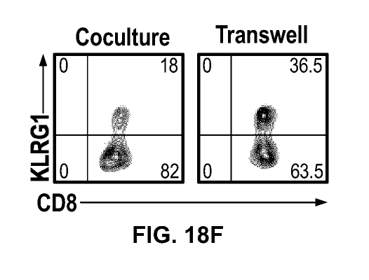

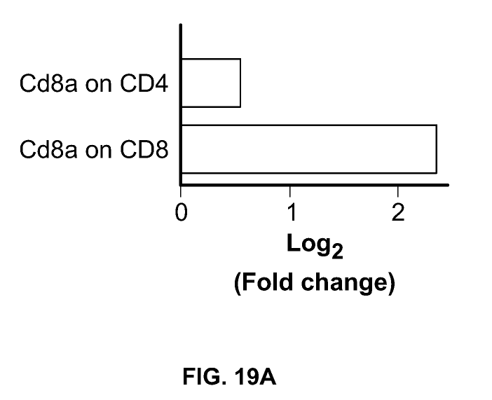

The transcriptomes of WT T9_{IL-33} versus ST2^{-/-}T9_{IL-33} cells were then compared in CD4 and CD8 sorted populations and found higher expression of cytolytic molecules (Gzma, Gzmb, Prf1, and Fas) as well as markers of the T-cell central memory phenotype (Cd621, Tcf7, and Cd27), which correlate with higher anti-tumor activity (FIG. 16B). Expression of cytolytic molecules was confirmed at the protein level, and perforin was abundant in Tc9_{IL-33} cells (FIG. 16C). Contact-dependent ST2/IL-33 signaling on CD4 T cells is crucial for higher granzyme-B and perforin expression by CD8 cells (FIG. 18A). Total WT T9_{IL-33} cells had significantly higher anti-tumor activity against MLL-AF9 AML than total T cells derived from ST2^{-/-}T9_{IL-33} or syngeneic T9_{IL-33} cells (FIG. 16D). CD8 and CD4 T cells sorted from WT T9_{IL-33} also showed higher specific anti-tumor activity than CD8 and CD4 T cells derived from ST2^{-/-} T9_{IL-33} or syngeneic T9_{IL-33} cells (FIG. 18B). CD8 polarized towards the Tc9_{IL-33} subset without CD4 T cells exhibited lower anti-tumoral activity than Tc9_{IL-33} cells in the presence of CD4 help (FIG. 18C). Ex-vivo analysis of bone marrow-infiltrating CD8 T cells showed that T9_{IL-33} cells expressed more granzyme-B and perforin (FIG. 16E). AML cells do not express EGFR (FIG. 18D), indicating that AREG does not affect the killing of leukemic cells and explaining its specificity for epithelial cells (FIG. 18E). T9_{IL-33} cells have a central memory phenotype (CD62L+CD44+CD27+KLRG1low) (FIG. 16F) that is mediated through ST2/IL-33 signaling on CD4 T cells (FIG. 18F). Analysis of CD8 T cells infiltrating bone marrow showed that more WT T9_{IL-33} cells retained their central memory phenotype compared to ST2^{-/-}T9_{IL-33} cells (FIG. 16G).

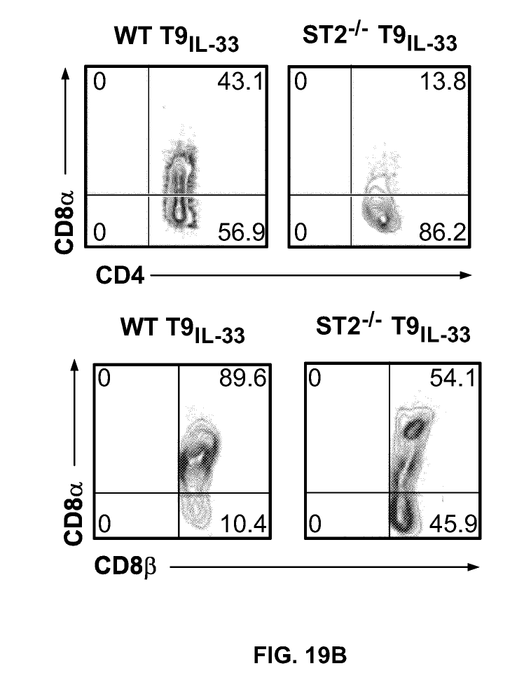

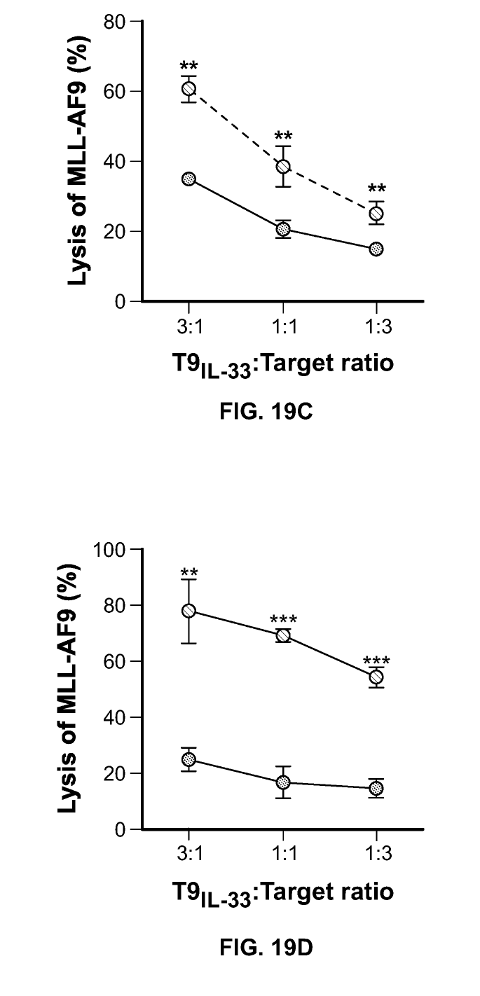

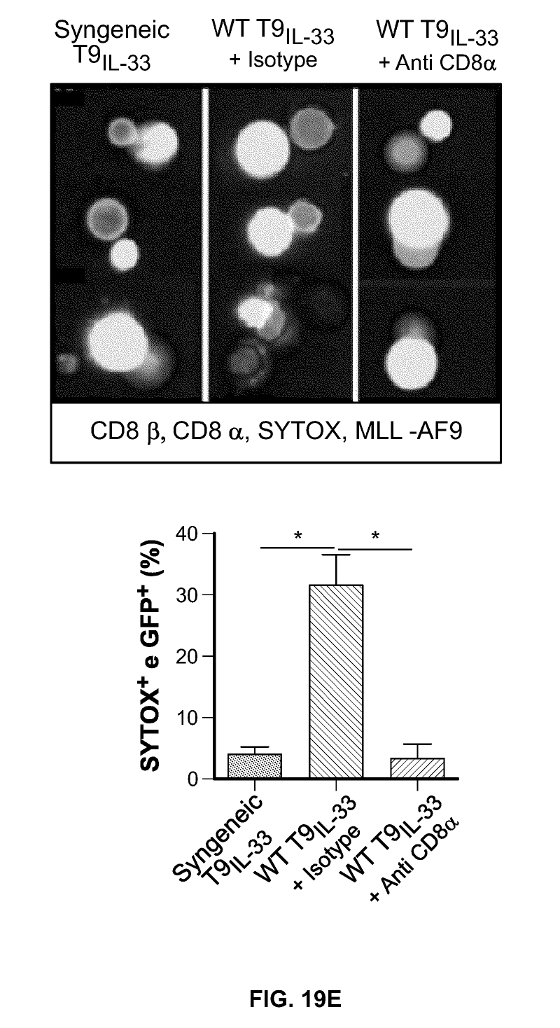

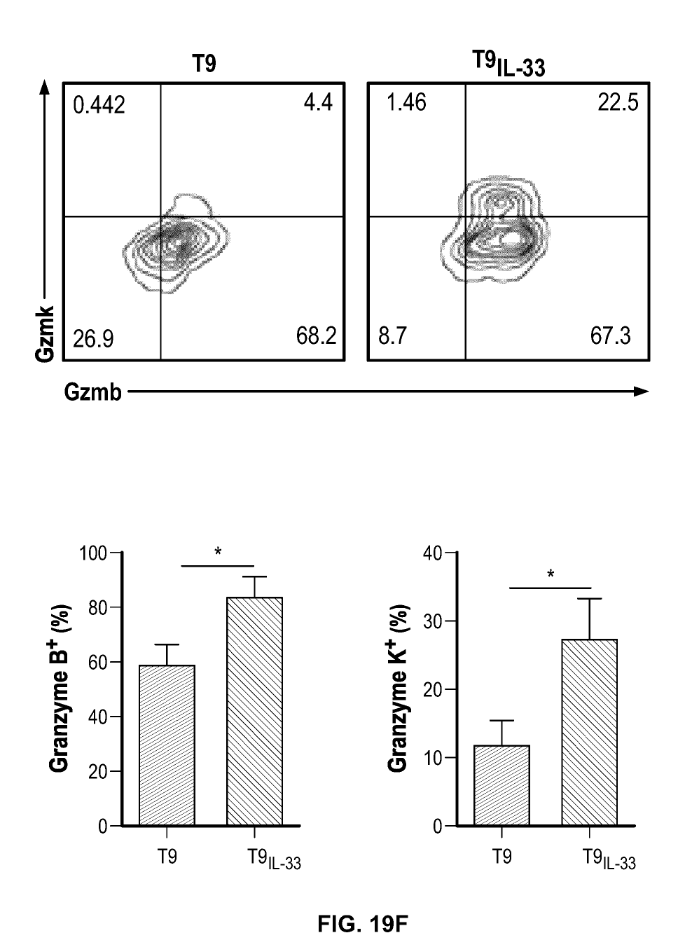

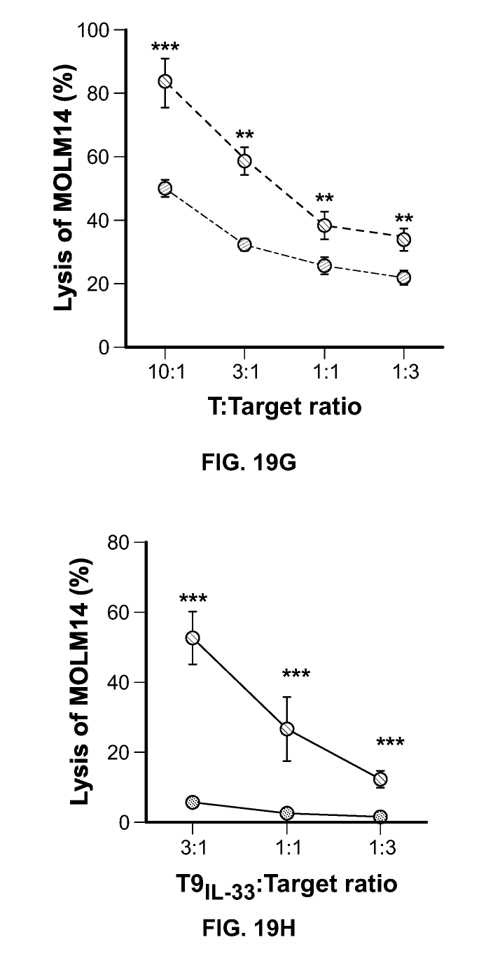

The possible mechanism of action was investigated through transcriptome analysis showing upregulation of CD8α expression at the mRNA and protein levels on both CD4 and CD8 cells sorted from WT T9_{IL-33} compared to ST2^{-/-}T9_{IL-33} cells (FIGS. 19A & 19B). Blocking CD8α during T9_{IL-33} differentiation reduced their cytolytic activity against MLL-AF9 cells (FIG. 19C). Likewise, blocking CD8α during cytolytic assays almost completely abolished the cytolytic activity of T9_{IL-33} (FIG. 19D). This observation signifies the importance of CD8α-expressing T9_{IL-33} cells in recognizing alloantigen on leukemic cells, and thus in triggering killing. The requirement of CD8α contact for tumor cell killing was confirmed by imaging studies in which T9_{IL-33} cells co-incubated with allogeneic MLL-AF9 cells showed SYTOX release, whereas syngeneic T9_{IL-33} cells or CD8α neutralized allogeneic T9_{IL-33} cells did not (FIG. 19E). Human T9 cells differentiated in the presence of IL-33 exhibited enhanced expression of granzymes B and K (FIG. 19F) as well as greater anti-tumoral cytolytic activity when incubated with MOLM14, an aggressive AML tumor cell line with FLT3/ITD mutations, as compared to T9 cells (FIG. 19G). Moreover, blocking CD8α during human T9IL-33 differentiation abolished their capacity to kill leukemia cells (FIG. 19H).

These Examples show that ST2/IL-33 activation of both murine and human IL-9-secreting T cells serves as a new T-cell therapy with dual opposing mechanisms: protecting normal tissues through upregulation of AREG and augmenting antitumor activity via CD8α upregulation. Thus, adoptive transfer of allogeneic T9_{IL-33} cells offer an attractive approach for not only separating GVT activity from GVHD, but also for generating autologous tumor-associated, antigen-specific T9_{IL-33} cells against leukemia or other malignancies.

What is claimed is:

1. A cell culture system comprising interleukin 4 (IL-4), transforming growth factor beta (TGFβ), interleukin-33 (IL-33), antibody to cluster of differentiation 3 (anti-CD3) and antibody of cluster of differentiation 28 (anti-CD28), and a cell.

2. The cell culture system as set forth in claim 1 comprising from about 5 ng/ml to about 100 ng/ml IL-4, from about 1 ng/ml to about 10 ng/ml TGFβ, from about 5 ng/ml to about 100 ng/ml IL-33, from about 1 μg/ml to about 10 μg/ml anti-CD28, and from about 0.5 μg/ml to about 5 μg/ml anti-CD3.

3. (canceled)

4. The cell culture system as set forth in claim 1 wherein the cell is selected from the group consisting of a peripheral blood mononuclear cell (PBMC) and a spleen cell.

5. The cell culture system as set forth in claim 4 wherein the cell is a PBMC, and wherein the PBMC is a lymphocyte.

6. The cell culture system as set forth in claim 5 wherein the lymphocyte is a T cell selected from the group consisting of a cluster of differentiation 4+ (CD4+) T cell and cluster of differentiation 8+ (CD8+) T cell.

7. The cell culture system as set forth in claim 6 wherein the T cell is selected from the group consisting of a T helper (Th9) cell and a T cytotoxic 9 (Tc9) cell.

8. The cell culture system as set forth in claim 6 wherein the T cell increased expression of ST2 and PU.1.

9. A method of cell culture for producing a T9IL-33 cell capable of producing cluster of differentiation 4+ (CD4+) and cluster of differentiation 8+ (CD8+) at frequencies of from about 10% to about 70% greater than a control T9 cell, the method comprising contacting a T9 cell with interleukin-33 (IL-33).

10. The method as set forth in claim 9 wherein the T9 cell is selected from the group consisting of T helper (Th9) cell and a T cytotoxic 9 (Tc9) cell.

11. The method as set forth in claim 9 wherein the T9 cell is contacted with from about 5 ng/ml to about 100 ng/ml IL-33.

12. A method of treating graft vs. host disease (GVHD), the method comprising administering to a subject in need thereof a cellular therapy comprising T9IL-33 cells.

13. The method of claim 12 wherein the subject has received allogeneic hematopoietic cell transplantation (allo-HCT).

14. A method of maintaining graft vs. leukemia activity in a subject in need thereof, the method comprising administering to the subject a cellular therapy comprising T9IL-33 cells.

15. The method of claim 14 wherein the subject has received allogeneic hematopoietic cell transplantation (allo-HCT).

16. The method of claim 14 wherein the subject has leukemia selected from the group consisting of acute myeloid leukemia (AML), acute lymphoblastic leukemia (ALL), and myeloid/lymphoid or mixed-lineage leukemia (MLL).

17. A method of treating a cancer, the method comprising administering to a subject in need thereof a cellular therapy comprising T9IL-33 cells.

18. The method of claim 17 wherein the subject has received allogeneic hematopoietic cell transplantation (allo-HCT).

19. The method of claim 17 wherein the subject has a hematological malignancy.

20. (canceled)

21. (canceled)

22. A T9IL-33 cell.

23. The T9IL-33 cell of claim 22 wherein the T9IL-33 cell expresses a cell surface marker selected from the group consisting of cluster of differentiation 8α (CD8α), suppression of tumorigenicity2 (ST2), interleukin-9 (IL-9), interleukin-10 (IL-10), Granzyme A (GrazA), Granzyme B (GrazB), cluster of differentiation 160 (CD160), Killer Cell Lectin-Like Receptor Subfamily K, Member 1 (KLRK1), cluster of differentiation 69 (CD69), cluster of differentiation (CD27), L-selectin (CD62L), CD45RO, CD45RA, Chemokine (C-C Motif) Receptor 7 (CCR7), and combinations thereof.

24. The T9IL-33 cell of claim 22 wherein the T9IL-33 cell does not express a cell surface marker selected from the group consisting of IFNγ and IL-4.


SEQUENCE LISTING (10 sequences)

SEQ ID NO: 1 - DNA, 24 bp, Artificial Sequence
  Synthetic
ctctggctcctagcaccatgaaga

SEQ ID NO: 2 - DNA, 26 bp, Artificial Sequence
  Synthetic
gtaaaacgcagctcagtaacagtccg

SEQ ID NO: 3 - DNA, 20 bp, Artificial Sequence
  Synthetic
aaggcacaccataaggctga

SEQ ID NO: 4 - DNA, 20 bp, Artificial Sequence
  Synthetic
tcgtagagcttgccatcgtt

SEQ ID NO: 5 - DNA, 22 bp, Artificial Sequence
  Synthetic
cacaaatgcaccttctgggaca

SEQ ID NO: 6 - DNA, 22 bp, Artificial Sequence
  Synthetic
tcactccaacgatacggtcctt

SEQ ID NO: 7 - DNA, 23 bp, Artificial Sequence
  Synthetic
ggacaatgcagggtaaaagttga

SEQ ID NO: 8 - DNA, 24 bp, Artificial Sequence
  Synthetic
tgaaagaaggaccaatgtcatttc

SEQ ID NO: 9 - DNA, 21 bp, Artificial Sequence
  Synthetic
ttggcctattcatgcgaagac

SEQ ID NO: 10 - DNA, 23 bp, Artificial Sequence
  Synthetic
gaggttccacgagctctctctct